(19) United States
(12) Patent Application Publication
(10) Pub. No.: US 2026/0250337 A1
(43) Pub. Date: Aug. 27, 2026

(54) HYBRID ANTIMICROBIAL PEPTIDES

(71) Applicants: UNIVERSIDADE FEDERAL DO ABC, São Paulo (BR); THE TRUSTEES OF THE UNIVERSITY OF PENNSYLVANIA, Philadelphia, PA (US)

(72) Inventors: César DE LA FUENTE-NUNEZ, Philadelphia, PA (US); Marcelo DER TOROSSIAN TORRES, Philadelphia, PA (US); Vani Xavier DE OLIVEIRA JUNIOR, Taboão da Serra (BR); Cibele NICOLASKI PEDRON, Santo André (BR)

(21) Appl. No.: 19/469,609

(22) PCT Filed: Mar. 26, 2024
(86) PCT No.: PCT/US2024/021479

§ 371 Date: Sep. 26, 2025

Related U.S. Application Data
(60) Provisional application No. 63/493,521, filed on Mar. 31, 2023.

(51) Int. Cl. C07K 14/435; A61K 38/00; A61P 31/04; C07K 14/47

(52) U.S. Cl. CPC C07K 14/43522; A61P 31/04; C07K 14/43568; C07K 14/4723; A61K 38/00; C07K 2319/55

(57) ABSTRACT

Provided herein are hybrid antimicrobial peptides, as well as methods for making such hybrid peptides, and methods treating microbial infections using the hybrid peptides.

### CROSS-REFERENCE TO RELATED APPLICATIONS

This application claims priority to U.S. Provisional App. No. 63/493,521, filed Mar. 31, 2023, the entire contents of which are incorporated herein by reference.

### GOVERNMENT RIGHTS

This invention was made with government support under GM138201 awarded by the National Institutes of Health and HDTRA1-21-1-0014 awarded by the Defense Threat Reduction Agency. The government has certain rights in the invention.

### TECHNICAL FIELD

The present disclosure pertains to the synthesis of antimicrobial peptides.

### BACKGROUND

The COVID-19 pandemic has made it apparent that infectious diseases are an ongoing threat. These diseases are caused by a variety of infectious agents including bacteria and eukaryotic parasites, as well as viruses, constituting leading causes of mortality worldwide. The widespread increase in antimicrobial-resistant bacterial infections is predicted to soon become a leading cause of morbidity and mortality in the world¹. The global burden of antimicrobial resistance is already well recognized, with a predicted number of deaths per year surpassing 1.27 million². Parasitic diseases, such as malaria, are also lethal, particularly in Africa, Asia, South America, and Central America. The World Health Organization (WHO) has estimated that malaria caused more than 409,000 deaths worldwide in 2019 alone³. Therefore, new strategies to treat infectious diseases are urgently needed. Peptides represent promising candidates⁴⁻⁶.

The biological activity of peptides can be harnessed for the development of antimicrobial drugs. Antimicrobial peptides (AMPs) are versatile molecules that present diverse mechanisms of action against pathogenic microorganisms, with the added advantage that, unlike conventional antibiotics, they do not readily select for bacterial resistance^{7, 8}. The activity of AMPs against eukaryotic cell types, such as parasites⁹ and cancer cells,¹⁰ is being explored because the physicochemical and structural features needed to exert these effects are tunable. These features can be enhanced by rational design, machine learning, and peptide engineering¹¹⁻¹⁴.

Most AMPs are cationic, small, and amphipathic¹⁵ and have broad-spectrum activity against pathogenic microorganisms; some, however, also exhibit cytotoxic activity against mammalian cells^{16, 17}. Most known AMPs tend to form α-helices upon contact with hydrophilic/hydrophobic interfaces, such as biological membranes, which are composed primarily of phospholipids¹⁴. Despite their promising activity profiles, several limitations have prevented the translation of naturally occurring AMPs into the clinic¹⁸, including cytotoxicity toward mammalian cells, instability, immunogenic effects, and high manufacturing costs¹⁹. In recent years, several approaches have been developed to optimize the design of synthetic peptides¹⁴ with improved biological activities and bioavailability, including computational methods¹² such as structure-activity relationship studies, neural networks, genetic and pattern recognition algorithms²⁰⁻²², and machine learning^{23, 24}, synthetic libraries²⁵; template-assisted methodologies²⁶; and sequence mutations²⁷. The most common techniques exploited for sequence mutations are amino acid substitutions^{28, 29} or deletions³⁰, design of truncated peptides^{31, 32}, and hybridization of peptides³³⁻³⁶ Hybridization, however, has not been as fully exploited as it might be, as a route to rational peptide design.

### SUMMARY

Disclosed herein are methods for preparing a hybrid antimicrobial peptide comprising selecting a parent peptide having antimicrobial activity and a portion that represents a helical structure when contacted with a cell membrane bilayer; selecting a template peptide having antimicrobial activity and a portion that represents a helical structure when contacted with a cell membrane bilayer; combining a hydrophilic face of the helical structure of the parent peptide with the hydrophobic face of the helical structure of the templated peptide, or combining a hydrophobic face of the helical structure of the parent peptide with the hydrophilic face of the helical structure of the template peptide, thereby forming a hybrid helical structure, wherein the hybrid antimicrobial peptide comprises the hybrid helical structure. Also disclosed are hybrid antimicrobial peptides produced by a such a method.

The present disclosure also provides hybrid antimicrobial peptides comprising a hybrid helical portion comprising a combination of a hydrophobic face of a parent antimicrobial peptide and a hydrophilic face of a template antimicrobial peptide, or a combination of a combination of a hydrophilic face of the parent antimicrobial peptide and a hydrophobic face of the template antimicrobial peptide.

Also disclosed are methods for treating a microbial infection in a subject comprising administering to the subject a hybrid antimicrobial peptide that is produced by a disclosed method, or a hybrid antimicrobial peptide as otherwise provided herein.

The present disclosure also provides methods for reducing a population of microbial organisms comprising contacting one or more of the microbial organisms with a hybrid antimicrobial peptide that is produced by a disclosed method, or a hybrid antimicrobial peptide as otherwise provided herein.

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

FIG. 1 provides a schematic of the design used to generate hybrid peptides and to evaluate their biological activity. The hydrophilic and hydrophobic faces of the helical structure of the AMPs temporin A, protonectin anoplin, and decoralin were combined with the hydrophilic and hydrophobic face of the AMP VmCT1 to generate hybrid peptides. The hybrid peptides were characterized to determine their secondary structure, stability in the presence of proteases and their in vitro and in vivo antimicrobial, hemolytic, and antiplasmodial activities, and for selected AMPs, their anticancer activities.

FIG. 2 provides a depiction of the hybrid peptide design strategy. Helical wheel projections of VmCT1, anoplin, protonectin, decoralin, temporin A and the hybrid peptides described in this study. The yellow circles indicate aromatic and aliphatic hydrophobic residues; gray circles correspond to residues with hydrophobicity close to zero; blue circles point to basic positively charged residues; purple circles represent polar uncharged residues; green circles represent the restricted pseudo amino acid proline; and pink circles represent polar uncharged amino acid residues. Black arrows indicate the direction and intensity of the hydrophobic moment¹⁰⁷. P and N indicate the hydrophilic and hydrophobic faces of the amphipathic projection, respectively.

FIG. 3 illustrates the antimicrobial, antiplasmodial, cytotoxicity, anticancer, and hemolytic activities of hAMPs and their parent AMPs. (a) Peptides in Peptone, Potato Dextrose, or Luria-Bertani broths were exposed to a fixed number of pathogenic (fungi, Gram-positive, and Gram-negative bacteria) cells (10⁶ CFU mL⁻¹) in a gradient of concentrations ranging (0-64 μmol L⁻¹), medium with no peptides and no bacteria were used as the control for microbial growth. Absorbance values colored in red represent maximal inhibition of microbial growth. On the contrary, blue corresponds to absorbance value reflective of growth (Anoplin-VmCT1: AV, Protonectin-VmVT1: PV, VmCT1-Protonectin: VP, Decoralin-VmCT1: DV, VmCT1-Decoralin: VD, Temporin A-VmCT1: TV, and VmCT1-Temporin A: VT). Anoplin, AV, Decoralin, DV and VD were not tested against the ESKAPE pathogens. MTT assay results for peptides after (b) 4 h (upper left panel) and (c) 24 h (upper right panel) using MCF-7 cancer cell lines. Dulbecco's Modified Eagle Medium was used as medium for the growth of MCF-7 and MCF-10A cell lines. Absorbance values in red are reflective of a decrease in viable MCF-7 cells, whereas blue correspond to cells that preserved viability. Experiments were performed in three independent replicates. (d) Values are expressed as the percentage of fluorescent mature sporozoites. Absorbance values in red represent active compounds against Plasmodium gallinaceum (fluorescent sporozoites), whereas absorbance values in blue indicate lack of antiplasmodial activity. Digitonin/PBS solution was used as the positive control and PBS solution alone was used as the negative control. (e) MTT assay results for healthy human breast epithelial cells MCF-10A lines exposed for 24 h to the peptides. Dulbecco's Modified Eagle Medium was used as the control for cell maximum growth. Again, in these experiments, blue represented viable MCF-10A cells, whereas red corresponded to cell death. Experiments were performed in three independent replicates. (f) Red blood cells were exposed for 1 h and at room temperature to peptides at concentrations ranging from 0.1 to 100 μmol L⁻¹), in PBS. Surfactant (1% SDS in PBS) was used to ensure complete hemolysis and PBS was used as a control preserving erythrocyte integrity. A PBS solution and the surfactant SDS were used as negative and positive controls, respectively. The MHC (maximal non-hemolytic concentration) was determined as the maximal concentration at which 100% of the erythrocytes were viable. All assays were done in three independent replicates.

FIG. 4 depicts a structural analysis of hAMPs and their interaction with lipid membranes. Circular dichroism (CD) spectra of the parent peptides and the hAMPs designed in this study in (a) water, (b) PBS (pH 7.4), (c) SDS (20 mmol L⁻¹), (d) POPC (10 mmol L⁻¹), (e) POPC:POPG (3:1, mol:mol-10 mmol L⁻¹), (f) POPC:DOPE (3:1, mol:mol-10 mmol L⁻¹) and (g) TFE/Water (3:2, v:v). CD were recorded after four accumulations at 20° C., using a 1 mm path length quartz cell, between 195 and 260 nm at 100 nm min⁻¹, with a band width of 0.5 nm. All assays were performed with peptides at 50 μmol L⁻¹. (h) Helical fractions (fu) values were calculated based on the Lifson-Roig model for each of the conditions analyzed (a-g). Initial configurations of AMP in (i) aqueous solution and (j) POPC:POPG bilayer membrane for MD simulations. Oxygen and hydrogen atom of water, POPC, POPG, Na⁺ and Clare colored red, white, blue, green, yellow, and cyan, respectively. The AMP VmCT1 in this example is colored according to the secondary structure determined in the Visual Molecular Dynamics (VMD) software (version 1.9.4). Note: the size of atoms in the system are scaled here for illustration and do not reflect their actual sizes in simulation. The experimental results for f_{H} obtained by CD experiments were compared with the ones obtained by MD simulations for the five parent peptides and the seven designed hAMPs in the (k) aqueous solution and (l) in lipid membrane. (m) Graph of the distance of center of mass of all the peptides studied to the membrane center in vertical (z) direction as a function of their f_{H} values. Light red colored region corresponds to a linear fit (black dashed line) from the peptides (except AV, VP and DV) with the uncertainty (standard deviation) of the linear slope and interception.

FIG. 5 depicts the results of an investigation into resistance to enzymatic degradation and mechanism of action of hAMPs. (a) Peptides were exposed to fetal bovine serum for 6 h. Aliquots were collected and analyzed by liquid chromatography. Three independent replicates were performed. (b) Graph of the mean square displacement (MSD) of VmCT1 peptide (V) as a function of time interval (Δt) in the lateral (XY plane) and vertical (Z axis) directions of the lipid membrane. (c) Calculation of isotropic and lateral self-diffusion coefficients (D) for all the peptides in this study in aqueous solution and lipid membrane from simulation, respectively. The diffusion coefficient of lateral diffusion of the peptides on lipid membrane is calculated from Eq. (1) based on the slope of the MSD profile with Δt<600 ns (vertical dashed line in b). (d) Damage and permeabilization of the bacterial outer membrane by the peptides using the NPN assay. (e) Effect of the peptides on the depolarization of the cytoplasmic membrane using the DiSC₃(5) assay.

FIG. 6 provides the results of an investigation into the anti-infective activity of AMPs and hAMPs in a skin abscess infection mouse model. (a) Anti-infective activity of VmCT1, Temporin A, VT, and TV peptides in a mouse model of A. baumannii skin infection as detailed in the methodology section. Statistical significance was determined using two-way ANOVA followed by Dunnett's test, **p<0.01 and ***p<0.001. (b) The toxicity of the peptide was monitored by mouse body weight measurements obtained during the experiment. Weights were normalized to the body weight of the mice at the beginning of the experiment, as a proxy for potential toxicity.

FIG. 7 provides a histopathological analysis of the murine skin tissue after the scarification procedure. The mice were shaved, and a scarification was performed with the tip of a needle on their back. Next, mice were (a) infected with A. baumannii or (b) left untreated. Samples were treated with either (c) VmCT1 (8 μmol L⁻¹), (d) TV (16 μmol L⁻¹), and (e) Polymyxin B (20 μmol L⁻¹).

FIGS. 8-19 provide chromatograms, mass spectroscopy, and mass scan results of respective AMPs/hAMPs.

### DETAILED DESCRIPTION OF ILLUSTRATIVE EMBODIMENTS

The present invention may be understood more readily by reference to the following detailed description taken in connection with the accompanying examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

The disclosures of each patent, patent application, and publication cited or described in this document are hereby incorporated herein by reference, in their entirety.

As employed above and throughout the disclosure, the following terms and abbreviations, unless otherwise indicated, shall be understood to have the following meanings.

In the present disclosure the singular forms “a”, “an”, and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. Furthermore, when indicating that a certain chemical moiety “may be” X, Y, or Z, it is not necessarily intended by such usage to exclude other choices for the moiety.

When values are expressed as approximations, by use of the antecedent “about,” it will be understood that the particular value forms another embodiment. As used herein, “about X” (where X is a numerical value) preferably refers to +10% of the recited value, inclusive. For example, the phrase “about 8” may refer to a value of 7.2 to 8.8, inclusive; as another example, the phrase “about 8%” may refer to a value of 7.2% to 8.8%, inclusive. Also, when the term “about” precedes a range, it is understood that the term modifies both recited endpoints and all points embraced within the range. For example, the phrase “about 1-10” is understood to mean “about 1 to about 10”, as well as “about x”, wherein x refers to any value between 1 and 10. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” In another example, when a listing of possible substituents including “hydrogen, alkyl, and aryl” is provided, the recited listing may be construed as including situations whereby any of “hydrogen, alkyl, and aryl” is negatively excluded; thus, a recitation of “hydrogen, alkyl, and aryl” may be construed as “hydrogen and aryl, but not alkyl”, or simply “wherein the substituent is not alkyl”.

Arachnids and insects are rich sources of amphipathic AMPs³⁷. Scorpions, for example, have several bioactive molecules in their venom, including AMPs³⁸. VmCT1, a linear helical AMP isolated from the venom of the scorpion Vaejovis mexicanus, has been reported to display antimicrobial and hemolytic activity³⁹. Examples of AMPs obtained from wasp venom with broad-spectrum antimicrobial activity³⁷ (defined here as activity against both Gram-positive and Gram-negative bacteria) include anoplin, isolated from the venom of Anoplius samariensis⁴⁰; protonectin, isolated from Agelaia pallipes, which has been reported as a non-hemolytic AMP⁴¹; and decoralin, which is derived from Oreumenes decorates and has anti-leishmania activity and low hemolytic activity⁴². Amphibians are also important sources of potent bioactive peptides⁴³, and AMPs that present antimicrobial⁴⁴, antiplasmodial⁴⁵, and anticancer activities⁴⁶ have been isolated from frog skin secretions. Temporin, derived from the skin of the European frog Rana temporaria, is a well-known AMPs that exhibits antimicrobial activity but minimal hemolytic activity^{47, 48}.

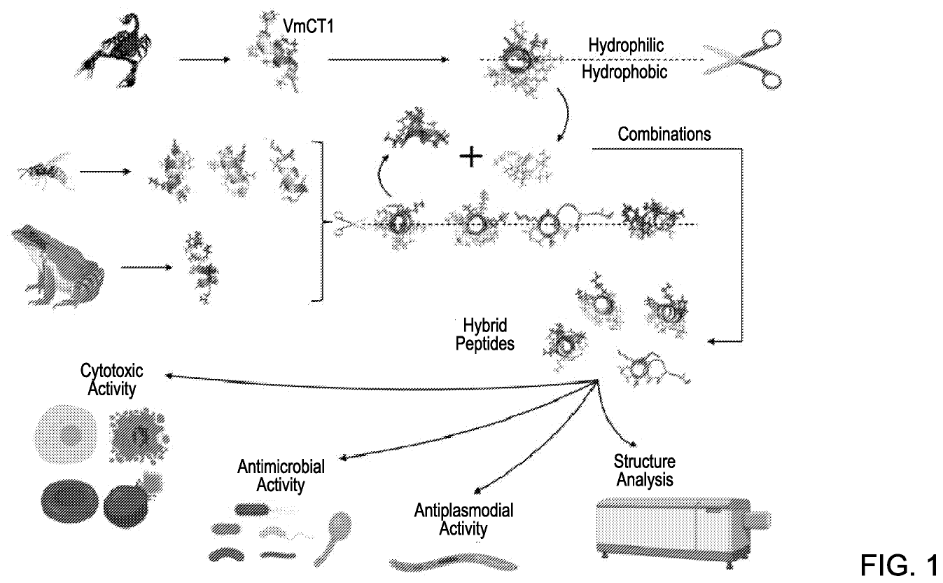

Although AMPs are represented by diverse peptide families and exert a range of biological activities, they have similar helical structures, lengths, and ratios of polar-to-nonpolar residues. Therefore, the present inventors selected them as templates for the formation of hybrid peptides. Hybrid peptides are synthetic molecules that contain amino acids or regions from two or more naturally occurring peptide sequences^{49, 50}. In peptide hybridization, such parts are combined to determine whether their biological activities are altered^{51, 52}. Pursuant to certain embodiments of the present invention, the hydrophilic and hydrophobic faces of the amphipathic helical structures of anoplin, protonectin, decoralin, and temporin A were combined with portions of the venom peptide VmCT1 to yield seven hybrid peptides. The venom peptide VmCT1 was selected as a design scaffold because of extensive structure-activity knowledge of this molecule⁵³. The hydrophilic and hydrophobic faces of VmCT1 were hybridized with amphipathic helical faces of anoplin, protonectin, decoralin, and temporin A to generate a series of highly active and non-toxic multifunctional peptides (FIG. 1). Peptide hybridization represents a promising molecular rearrangement strategy for optimizing the antimicrobial and antiplasmodial properties of AMPs while minimizing their cytotoxicity.

Accordingly, disclosed herein are methods for preparing a hybrid antimicrobial peptide comprising selecting a parent peptide having antimicrobial activity and a portion that represents a helical structure when contacted with a cell membrane bilayer; selecting a template peptide having antimicrobial activity and a portion that represents a helical structure when contacted with a cell membrane bilayer; combining a hydrophilic face of the helical structure of the parent peptide with the hydrophobic face of the helical structure of the templated peptide, or combining a hydrophobic face of the helical structure of the parent peptide with the hydrophilic face of the helical structure of the template peptide, thereby forming a hybrid helical structure, wherein the hybrid antimicrobial peptide comprises the hybrid helical structure.

The template peptide may be selected by assessing one or more of the amphiphilicity, antimicrobial activity, and hemolytic activity of respective candidate peptides from a population of candidate peptides. Candidate peptides with high amphiphilicity and antimicrobial activity, and with low hemolytic activity can represent preferred choices for the template peptide. Candidate template peptides may be selected from known antimicrobial peptides, such as those that are derived from animal (e.g., insect, arachnid, or reptile) venom. Likewise parent peptides may be selected from known antimicrobial peptides, such as those that are derived from animal (e.g., insect, arachnid, or reptile) venom.

The present methods may further comprise the step of aligning the size of the hydrophobic face of the hybrid helical structure with the size of the hydrophilic face of the hybrid helical structure. One objective of such aligning can be characterized as providing a standardized amino acid length for both the hydrophilic face hydrophobic face of the hybrid helical structure and the hydrophilic face of the hybrid helical structure. The aligning may comprise adding one or more amino acid residues to an interface between the hydrophobic face of the hybrid helical structure and the hydrophilic face of the hybrid helical structure, for example, in order to provide the standardized amino acid length. For example, aligning may comprise adding one or more glycine residues to an interface between the hydrophobic face of the hybrid helical structure and the hydrophilic face of the hybrid helical structure, for example, in order to provide the standardized amino acid length.

To the extent that aligning or other adjustments results in a standardized amino acid length with respect to the hybrid helical structure, the standardized amino acid length may correspond to the number of amino acids of the parent peptide, such as of the helical portion of the parent peptide.

In some embodiments, the parent peptide is VmCT1. In certain embodiments, the template peptide is anoplin, protonectin, decoralin, or temporin A.

Also disclosed are hybrid antimicrobial peptides produced by any of embodiment of the above-described inventive method.

The present disclosure also provides hybrid antimicrobial peptides comprising a hybrid helical portion comprising a combination of a hydrophobic face of a parent antimicrobial peptide and a hydrophilic face of a template antimicrobial peptide, or a combination of a combination of a hydrophilic face of the parent antimicrobial peptide and a hydrophobic face of the template antimicrobial peptide. In some embodiments, the hybrid helical portion comprises one or more introduced spacer amino acids for aligning by size the hydrophobic face with the hydrophilic face. The spacer amino acids may be positioned, for example, at an interface between the hydrophobic face and the hydrophilic face. The hydrophobic face and hydrophilic face may respectively be derived from parent and template antimicrobial peptides using the selection criteria described supra.

Also disclosed are methods for treating a microbial infection in a subject comprising administering to the subject a hybrid antimicrobial peptide that is produced by a disclosed method, or a hybrid antimicrobial peptide as otherwise provided herein.

The hybrid antimicrobial peptides according to the present disclosure may be provided in a composition that is formulated for any type of administration. For example, the compositions may be formulated for administration orally, topically, parenterally, enterally, or by inhalation (e.g., intranasally). The active agent may be formulated for neat administration, or in combination with conventional pharmaceutical carriers, diluents, or excipients, which may be liquid or solid. The applicable solid carrier, diluent, or excipient may function as, among other things, a binder, disintegrant, filler, lubricant, glidant, compression aid, processing aid, color, sweetener, preservative, suspensing/dispersing agent, tablet-disintegrating agent, encapsulating material, film former or coating, flavoring agent, or printing ink. Any material used in preparing any dosage unit form is preferably pharmaceutically pure and substantially non-toxic in the amounts employed. In addition, the active agent may be incorporated into sustained-release preparations and formulations. Administration in this respect includes administration by, inter alia, the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol, and rectal systemic.

In powders, the carrier, diluent, or excipient may be a finely divided solid that is in admixture with the finely divided active ingredient. In tablets, the active ingredient is mixed with a carrier, diluent or excipient having the necessary compression properties in suitable proportions and compacted in the shape and size desired. For oral therapeutic administration, the active compound may be incorporated with the carrier, diluent, or excipient and used in the form of ingestible tablets, buccal tablets, troches, capsules, elixirs, suspensions, syrups, wafers, and the like. The amount of active agent(s) in such therapeutically useful compositions is preferably such that a suitable dosage will be obtained.

Liquid carriers, diluents, or excipients may be used in preparing solutions, suspensions, emulsions, syrups, elixirs, and the like. The active ingredient of this invention can be dissolved or suspended in a pharmaceutically acceptable liquid such as water, an organic solvent, a mixture of both, or pharmaceutically acceptable oils or fat. The liquid carrier, excipient, or diluent can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators.

Suitable solid carriers, diluents, and excipients may include, for example, calcium phosphate, silicon dioxide, magnesium stearate, talc, sugars, lactose, dextrin, starch, gelatin, cellulose, methyl cellulose, ethylcellulose, sodium carboxymethyl cellulose, microcrystalline cellulose, polyvinylpyrrolidine, low melting waxes, ion exchange resins, croscarmellose carbon, acacia, pregelatinized starch, crospovidone, HPMC, povidone, titanium dioxide, polycrystalline cellulose, aluminum methahydroxide, agar-agar, tragacanth, or mixtures thereof.

Suitable examples of liquid carriers, diluents and excipients, for example, for oral, topical, or parenteral administration, include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil), or mixtures thereof.

For parenteral administration, the carrier, diluent, or excipient can also be an oily ester such as ethyl oleate and isopropyl myristate. Also contemplated are sterile liquid carriers, diluents, or excipients, which are used in sterile liquid form compositions for parenteral administration. Solutions of the active agents can be prepared in water suitably mixed with a surfactant, such as hydroxypropylcellulose. A dispersion can also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier, diluent, or excipient may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In some instances, the antimicrobial peptides themselves may be sufficient to prevent contamination by microorganisms. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active agent in the pharmaceutically appropriate amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and freeze drying techniques that yield a powder of the active ingredient or ingredients, plus any additional desired ingredient from the previously sterile-filtered solution thereof.

Thus, an antimicrobial peptide may be in the present compositions and methods in an effective amount by any of the conventional techniques well-established in the medical field. For example, the administration may be in the amount of about 0.1 mg/day to about 500 mg per day. In some embodiments, the administration may be in the amount of about 250 mg/kg/day. Thus, administration may be in the amount of about 0.1 mg/day, about 0.5 mg/day, about 1.0 mg/day, about 5 mg/day, about 10 mg/day, about 20 mg/day, about 50 mg/day, about 100 mg/day, about 200 mg/day, about 250 mg/day, about 300 mg/day, or about 500 mg/day.

The present disclosure also provides methods for reducing a population of microbial organisms comprising contacting one or more of the microbial organisms with a hybrid antimicrobial peptide that is produced by a disclosed method, or a hybrid antimicrobial peptide as otherwise provided herein.

In accordance with the methods of reducing a population of microbial organisms comprising contacting one or more of the microbial organisms with a hybrid antimicrobial peptide that is produced by a disclosed method, or a hybrid antimicrobial peptide as otherwise provided herein, microbes against which the present hybrid antimicrobial peptides are effective may be, for example, any unicellular organism, such as gram-negative bacteria, gram-positive bacteria, protozoa, viruses, bacteriophages, and archaea. The present hybrid peptides can have an antimicrobial effect with respect to any such microbe.

Examples of bacteria against which the present hybrid peptides are effective to cause reduction in numbers include gram positive bacteria and gram negative bacteria, for example, Salmonella enterica, Listeria monocytogenes, Escherichia coli, Clostridium botulinum, Clostridium difficile, Campylobacter, Bacillus cereus, Vibrio parahaemolyticus, Vibrio cholerae, Vibrio vulnificus, Staphylococcus aureus, Yersinia enterocolitica, Shigella, Moraxella spp., Helicobacter, Stenotrophomonas, Bdellovibrio, Legionella spp. (e.g., pneumophila), Neisseria gonorrhoeae, Neisseria meningitidis, Haemophilus influenzae, Acinetobacter baumannii, Klebsiella pneumoniae, Pseudomonas aeruginosa, Proteus mirabilis, Enterobacter cloacae, Serratia marcescens, Helicobacter pylori, Salmonella enteritidis, Salmonella typhi, and combinations thereof. Examples of Salmonella enterica serovars that can be reduced using the compounds of the disclosure include, for example, Salmonella enteriditis, Salmonella typhimurium, Salmonella poona, Salmonella heidelberg, and Salmonella anatum. Exemplary viruses against which the present peptides are effective to cause reduction in numbers include coronaviruses, rhinoviruses, and influenza viruses.

Hereinafter, the present disclosure will be described in more detail through Examples, which are intended to be illustrative to the present disclosure, although present disclosure is not limited to the Examples.

### Example 1—Design of Hybrid Antimicrobial Peptides (hAMPs)

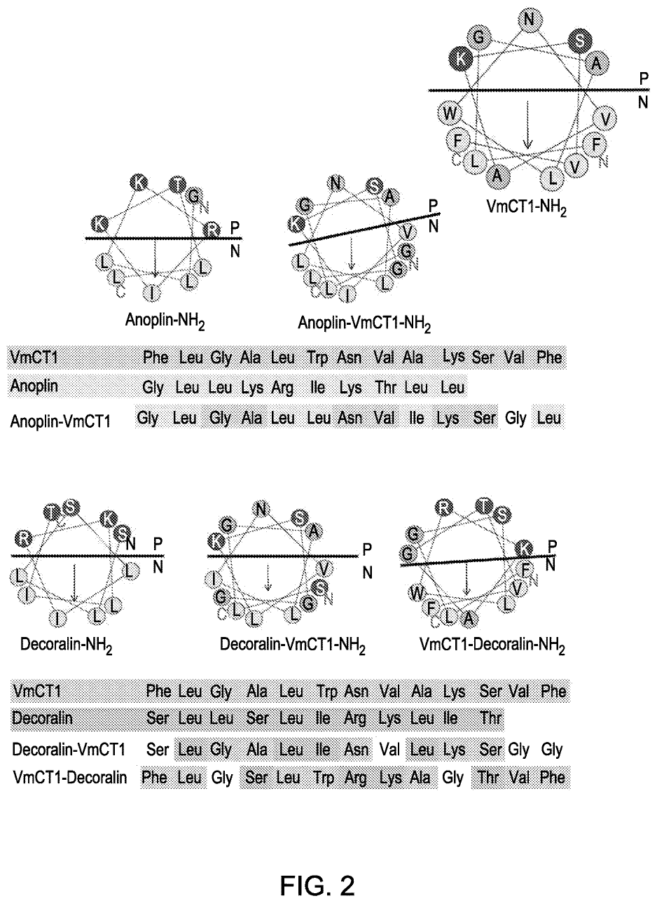

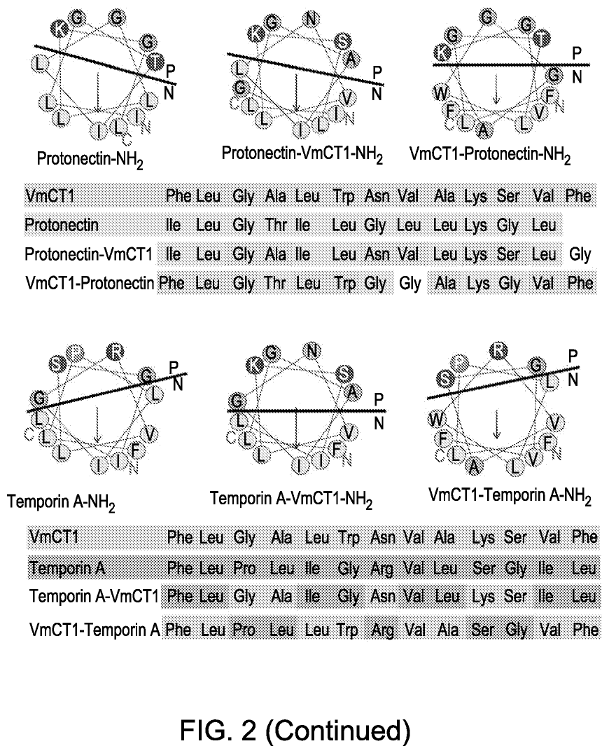

Seven hybrid AMPs (hAMPs) derived from VmCT1, a highly active broad-spectrum AMP, and four naturally occurring AMP templates were designed and their biological activities were investigated. To design the hAMPs, we first used the HeliQuest server to predict the helical wheel projection since all the selected parent AMPs are known to be helical upon contact with membrane bilayers. The server considers a perfect helical disposition (100° between amino acids) and projects the sequence in two dimensions. We used those projections to curate which amino acid residues are part of the hydrophilic and hydrophobic faces of the helical structure (FIG. 2). Next, we combined the hydrophilic face of the main parent AMP VmCT1 with the hydrophobic face of one of the other four parent AMPs and vice-versa (FIG. 2 and Table 1). In addition to their helical secondary structure, the template peptides (anoplin, protonectin, decoralin, and temporin A) were also selected based on their amphiphilicity, potent antimicrobial activity, and low hemolytic activity^{40, 42, 48, 54}. These AMPs have slightly different numbers of amino acid residues in their sequences; therefore, we added Gly residues (the simplest side chain group and commonly used as a spacer⁵⁵) to the hydrophobic/hydrophilic interfaces, i.e., the frontier between the hydrophobic and hydrophilic faces in the helical wheel projection for each hAMP, to align sequence sizes in a pairwise manner. Thus, all designed hAMP sequence sizes were standardized to a total of 13 amino acid residues, a size equivalent to that of the template molecule VmCT1.

TABLE 1
Peptides sequence, molecular characterization, and physiochemical properties ¹⁰⁷.

| | | | Molecular Weight (Da) | | | | | |
|---|---|---|---|---|---|---|---|---|
| Label | Peptide | Sequence* | Theoretical | Observed | H | μH | z | P/N |
| V | VmCT1 | Phe-Leu-Gly-Ala-Len-Trp-Asu-Val-Ala- | 1450.7 | 1451 | 0.82 | 0.58 | +2 | 0.50 |
| | | Lys-Ser-Val-Phe | | | | | | |
| A | Anoplin | 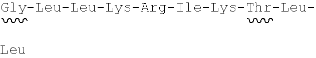 | 1152.5 | 1153 | 0.59 | 0.71 | +4 | 1.00 |
| AV | Anoplin- | Gly-Leu-Gly-Ala-Leu-Len-Asn-Val-Ile- | 1253.5 | 1254 | 0.65 | 0.55 | +2 | 0.86 |
| | VmCT1 | Lys-Ser-Gly-Leu | | | | | | |
| P | 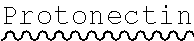 | 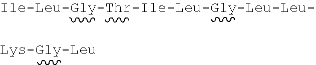 | 1209.6 | 1210 | 0.95 | 0.68 | +2 | 0.71 |
| PV | Protonectin- | Ile-Leu-Gly-Ala-Ile-Leu-Asn-Val-Leu- | 1309.6 | 1310 | 0.77 | 0.66 | +2 | 0.62 |
| | VmCT1 | Lys-Ser-Leu-Gly | | | | | | |
| VP | VmCTI- | 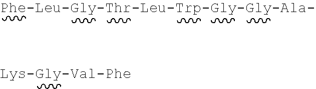 | 1351.6 | 1351 | 0.77 | 0.52 | +2 | 0.86 |
| | Protonectin | | | | | | | |
| D | 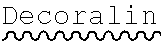 | Ser-Leu-Leu-Ser-Len-Ile-Arg- | 1254.8 | 1256 | 0.78 | 0.65 | +3 | 0.83 |
| | | Lys-Leu-Ile-Thr | | | | | | |
| DV | Decoralin- | Ser-Leu-Gly-Ala-Leu-Ile-Asn-Val-Leu- | 1227.4 | 1229 | 0.52 | 0.46 | +2 | 1.16 |
| | VmCT1 | Lys-Ser-Gly-Gly | | | | | | |
| VD | VmCTI- | 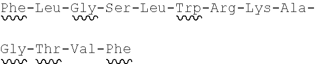 | 1480.7 | 1481 | 0.69 | 0.58 | +4 | 0.86 |
| | Decoralin | | | | | | | |
| T | Temporin A | Phe-Leu-Pro-Leu-Ile-Gly-Arg-Val-Leu- | 1396.8 | 1397 | 1.00 | 0.61 | +2 | 0.44 |
| | | Ser-Gly-Ile-Leu | | | | | | |
| TV | Temporin A- | Phe-Leu-Gly-Ala-Ile-Gly-Asn-Val-Leu- | 1343.7 | 1344 | 0.80 | 0.71 | +2 | 0.62 |
| | VmCTI | Lys-Ser-Ile-Leu | | | | | | |
| VT | VmCT1- | Phe-Leu-Pro-Leu-Leu-Trp-Arg-Val-Ala- | 1503.8 | 1504 | 1.00 | 0.47 | +2 | 0.30 |
| | Temporin A | Ser-Gly-Val-Phe | | | | | | |

In Table 1, all sequences present amidated C-terminus. H (hydrophobicity), μ_{H} (hydrophobic moment), z (net charge), P/N (ratio of polar/non-polar residues in the sequence) according to helical wheel projection server Heliquest¹⁰⁷. Mass obtained under the following conditions: Phenomenex Gemini C18 column (2.0 mm×150 mm, 3.0 μm particles, 110 Å pores). Solvent A was 0.1% TFA in water, and solvent B was 90% ACN in solvent A. Elution with a 5-95% B gradient was performed over 20 min, 0.2 mL min⁻¹ flow and peptides were detected at 220 nm. Mass measurements were performed in a positive mode with the following conditions: mass range between 100 to 2500 m/z, ion energy of 5.0 V, nitrogen gas flow of 12.0 L min⁻¹, solvent heater of 250° C., multiplier of 1.0, capillary of 3.0 kV and cone voltage of 35 V.

By choosing this standard size, it was possible to avoid the insertion of hydrophobic residues from the template peptides (anoplin, protonectin, decoralin, and temporin) into the hydrophilic face of VmCT1, as well as the need to insert hydrophilic residues from the other peptides into the hydrophobic face of VmCT1 (FIG. 2). The designed hybrid peptides were designated as: AV, VP, PV, VD, DV, VT, and TV (A is anoplin; Vis VmCT1; P is protonectin; D is decoralin; and T is temporin A). For each hybrid peptide acronym, the first letter refers to the hydrophilic face whereas the second letter indicates the hydrophobic face.

Not all hybridization combinations could be synthesized using our methodology. For example, we were unable to synthesize peptide VA combining the hydrophilic portion of VmCT1 with the hydrophobic portion of anoplin, although we attempted various solid-phase synthesis and manual synthesis strategies using conditions such as a range of coupling agents [e.g., N,N,N′,N′-tetramethyl-O-(1H-benzotriazol-1-yl) uronium hexafluorophosphate, 2-(1H-Benzotriazole-1-yl)-1,1,3,3-tetramethylaminium tetrafluoroborate, hydroxybenzotriazole] and coupling reaction times (e.g., 30-120 minutes at room temperature).

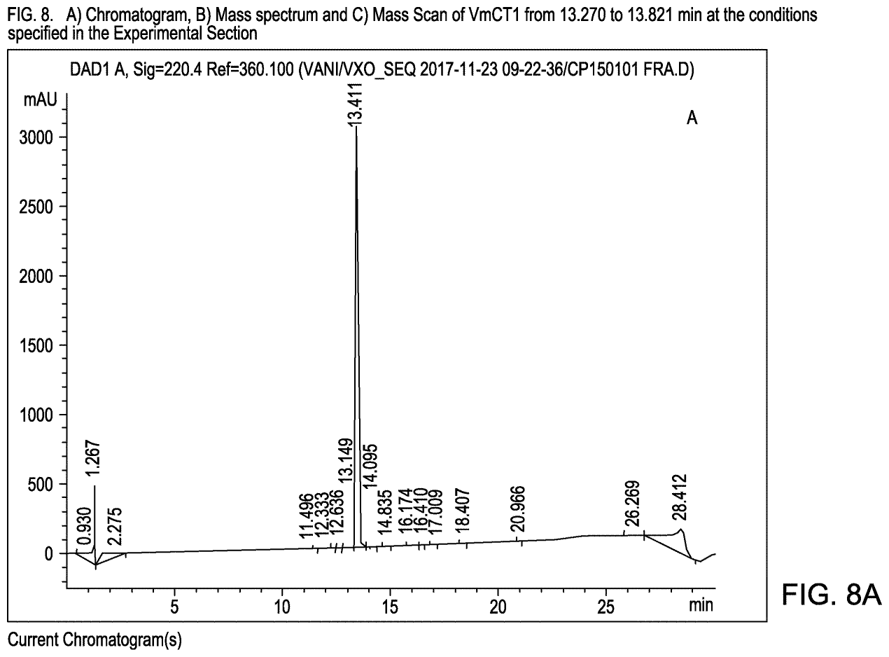

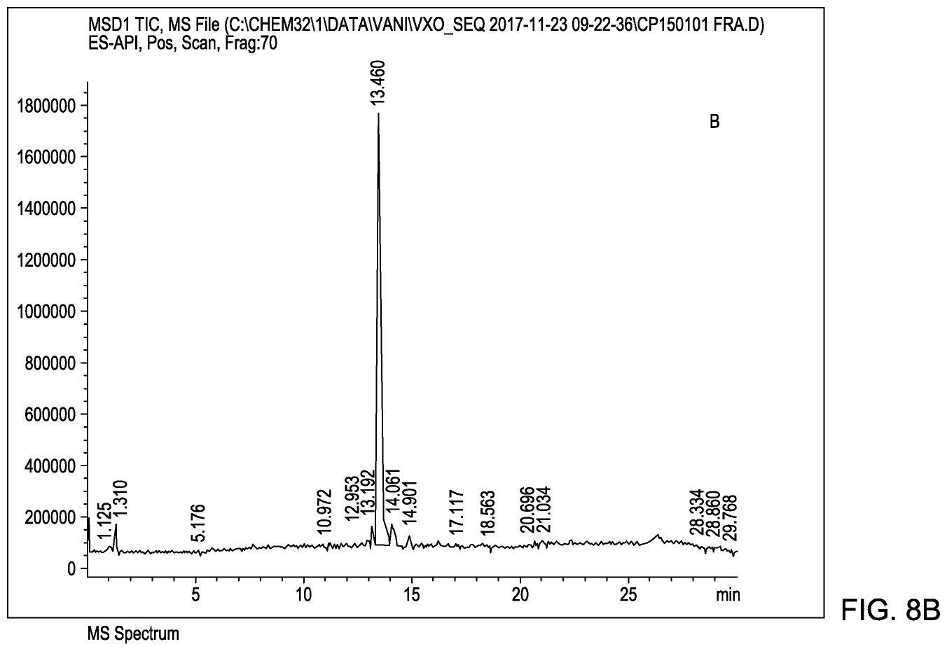

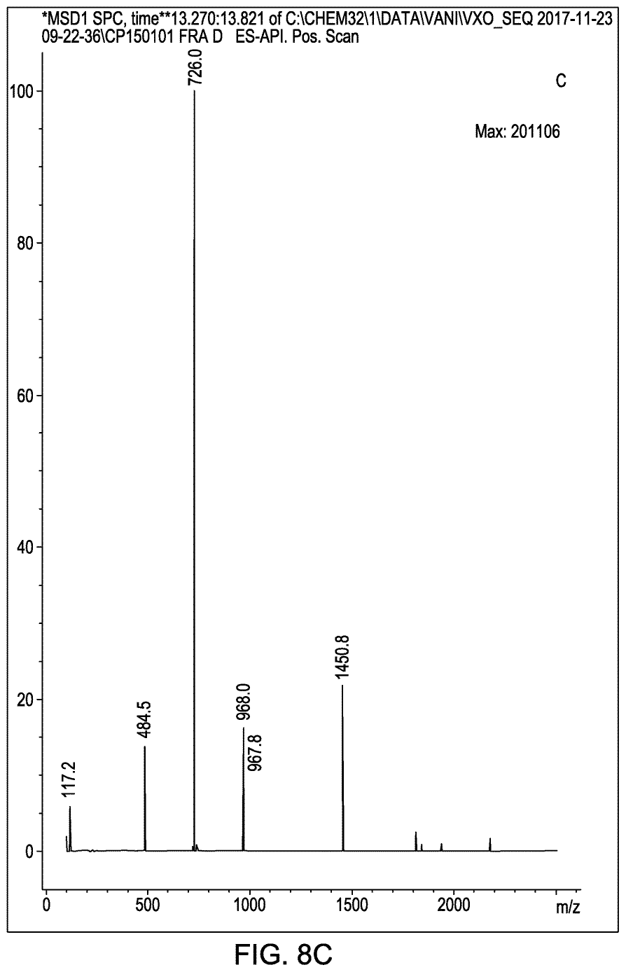

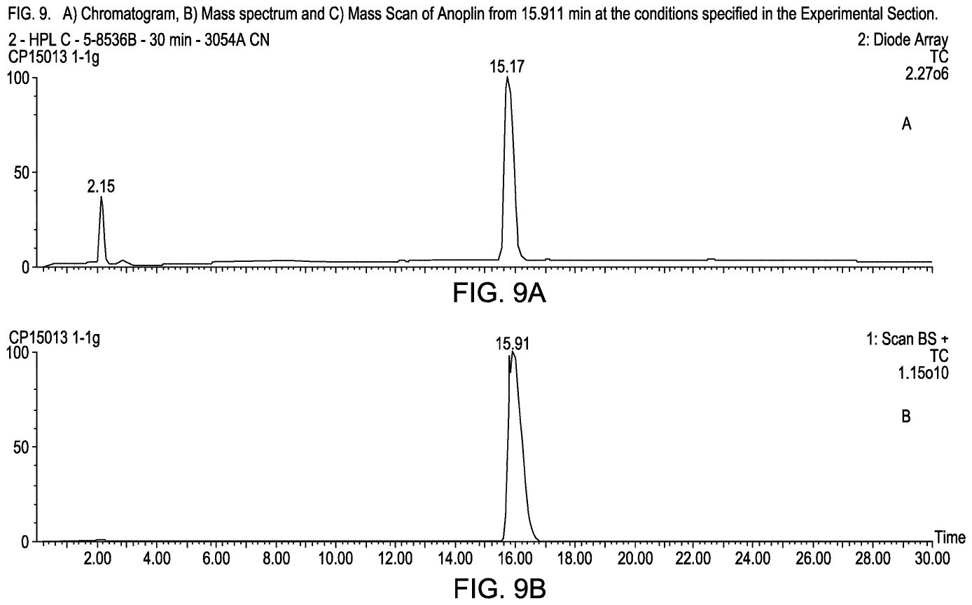

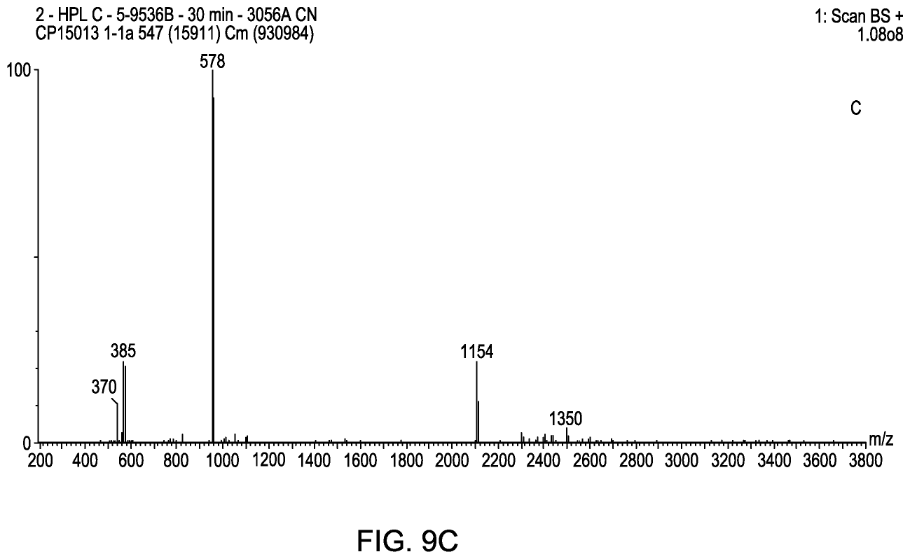

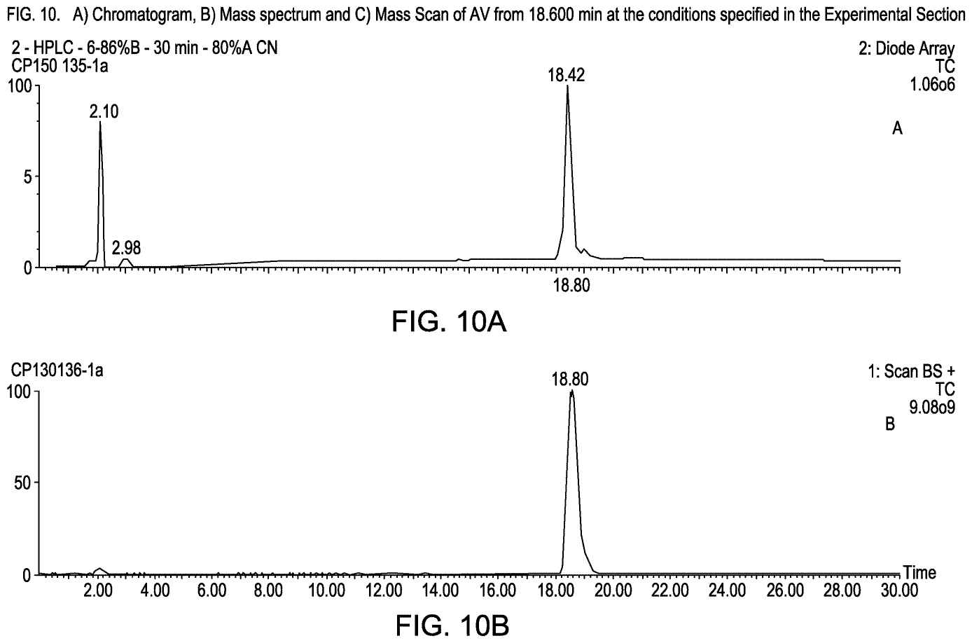

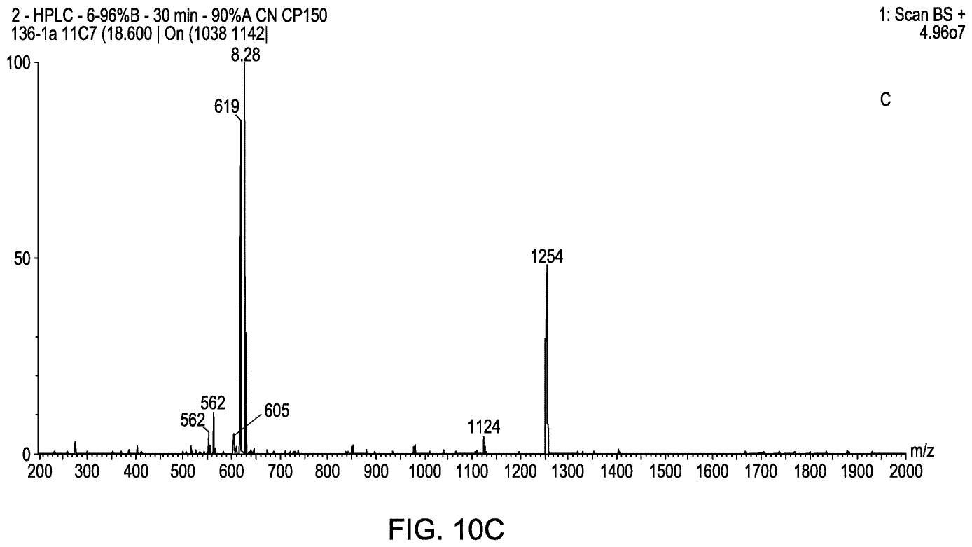

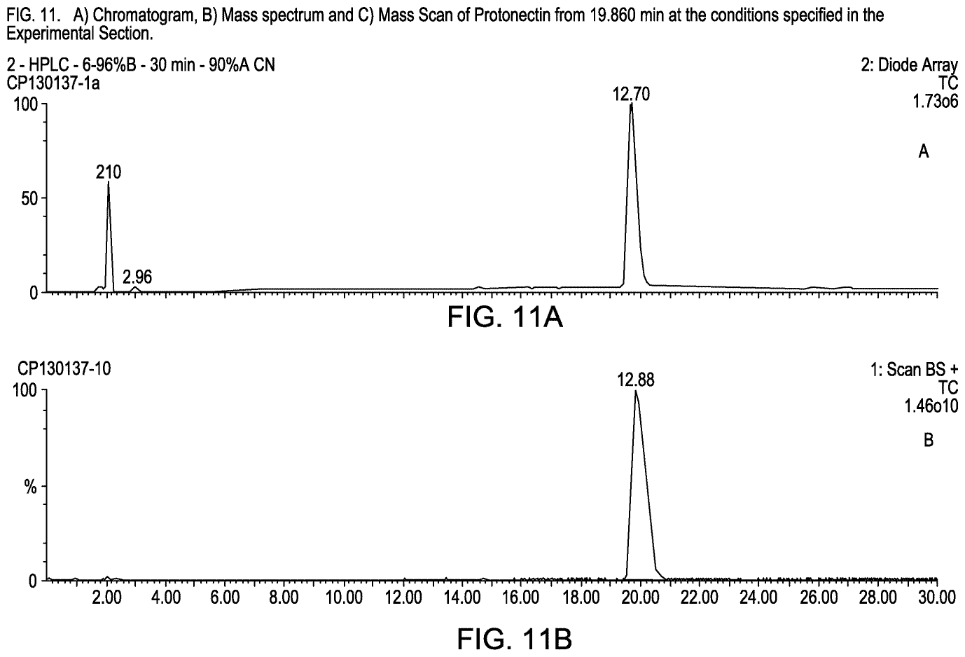

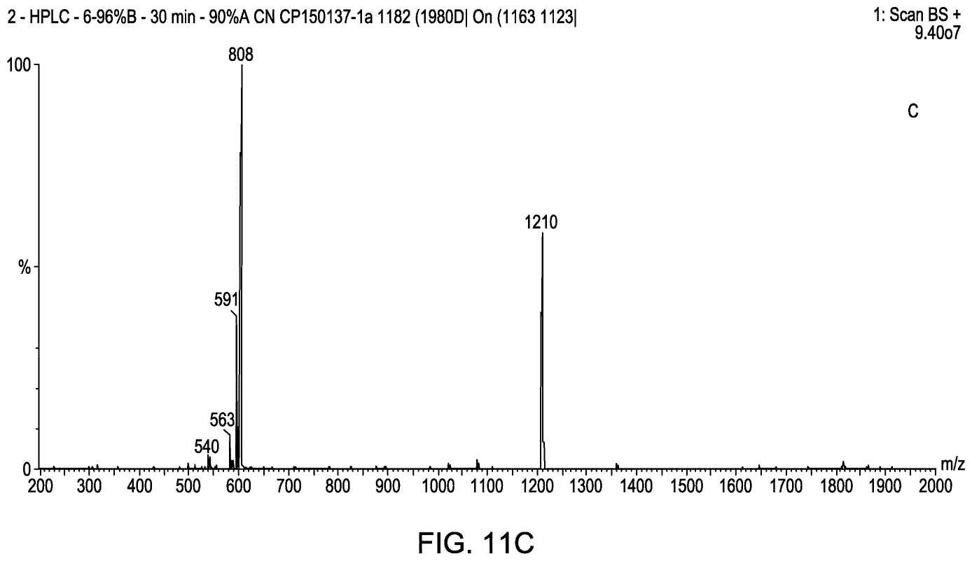

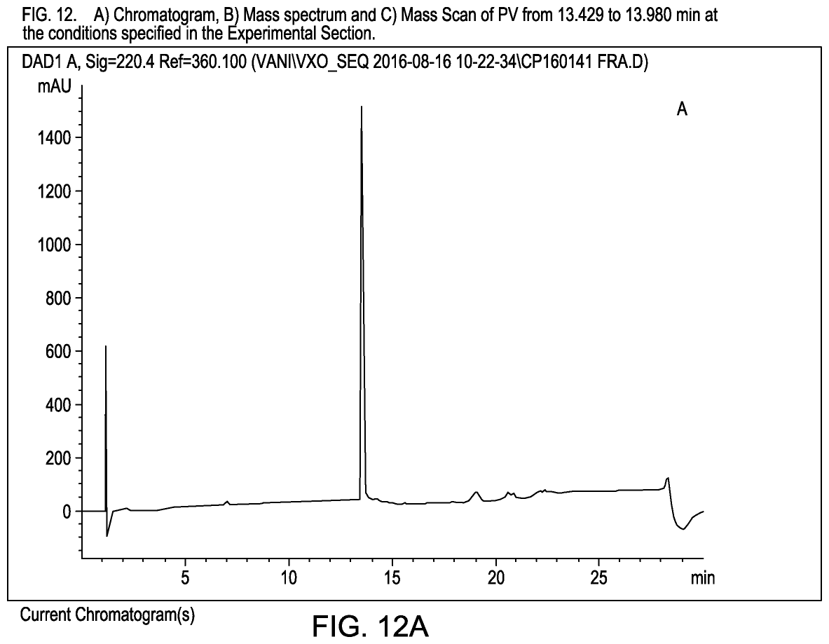

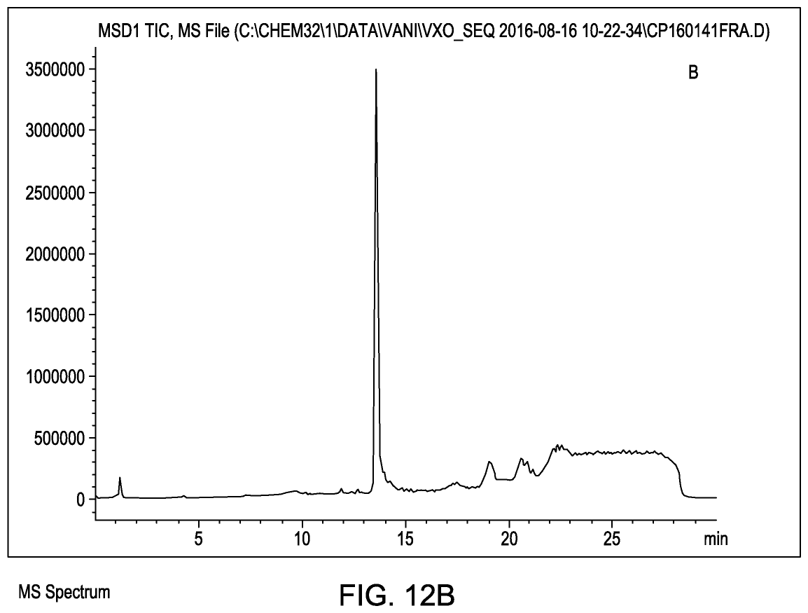

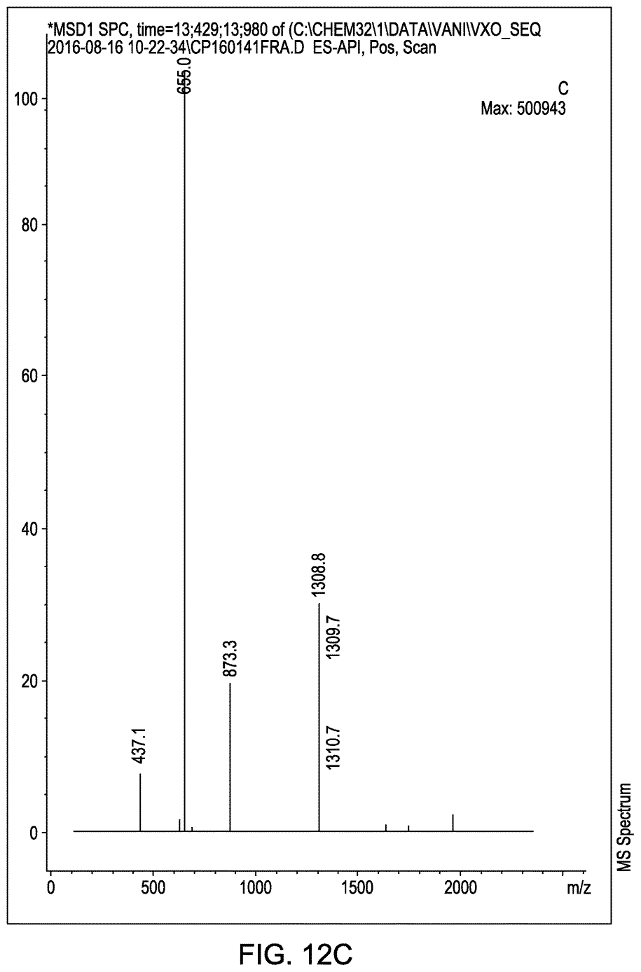

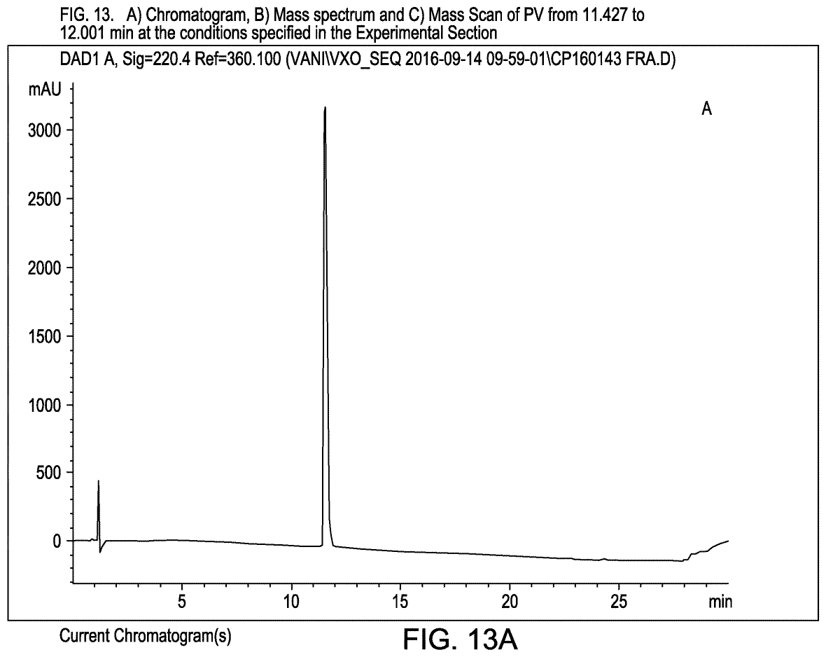

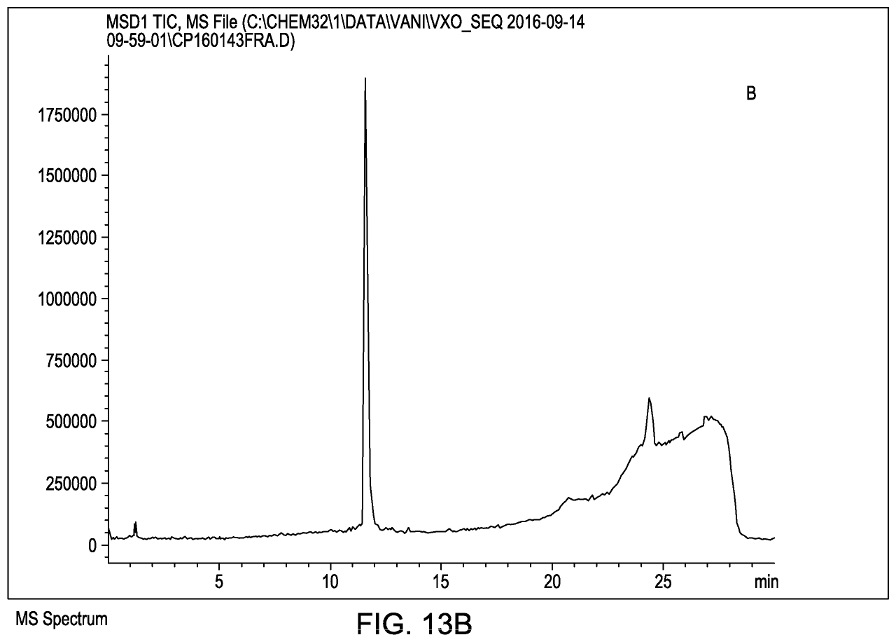

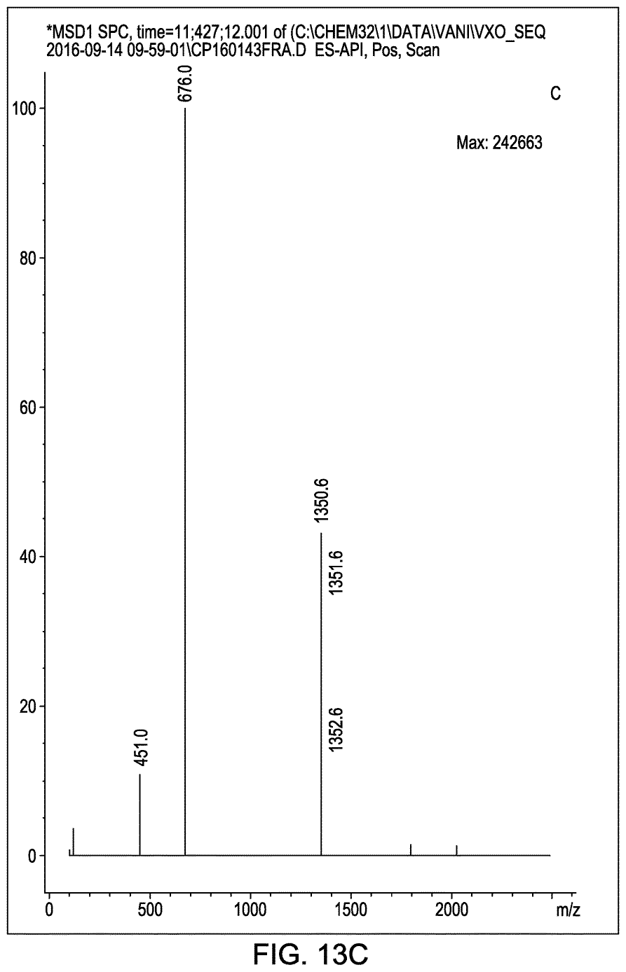

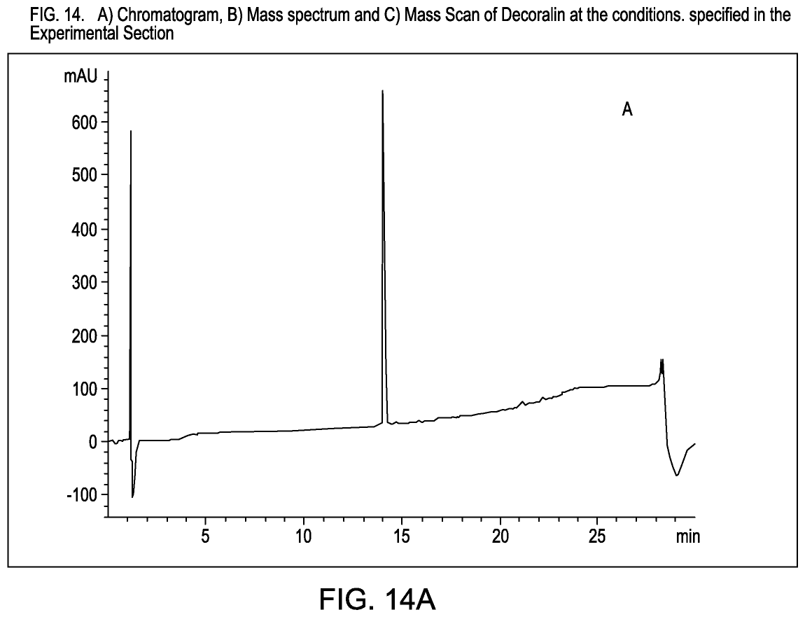

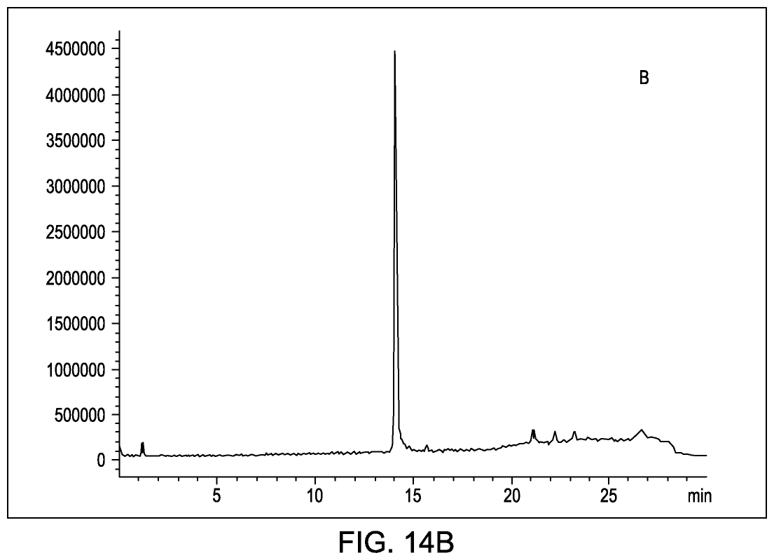

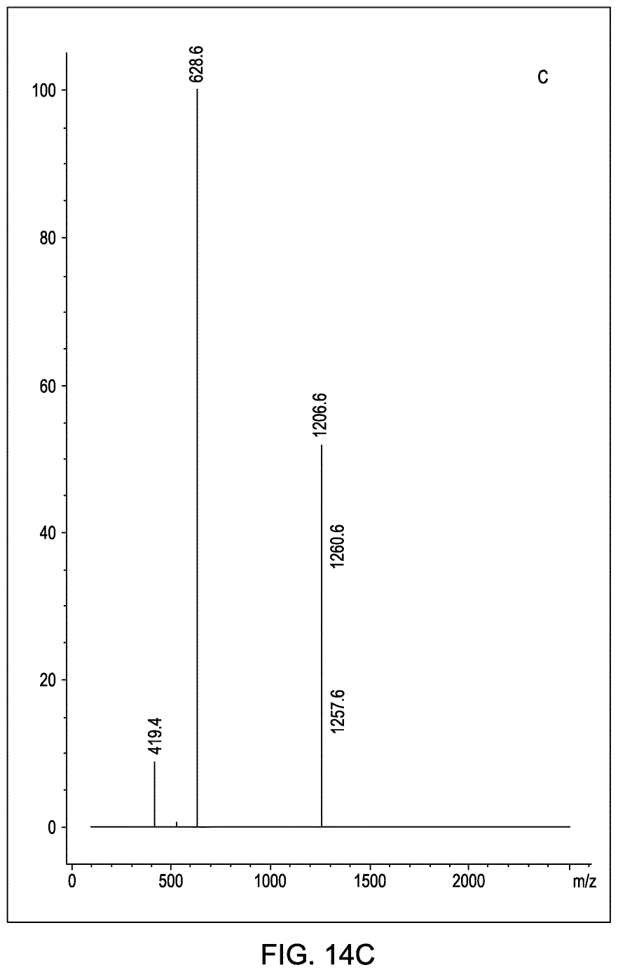

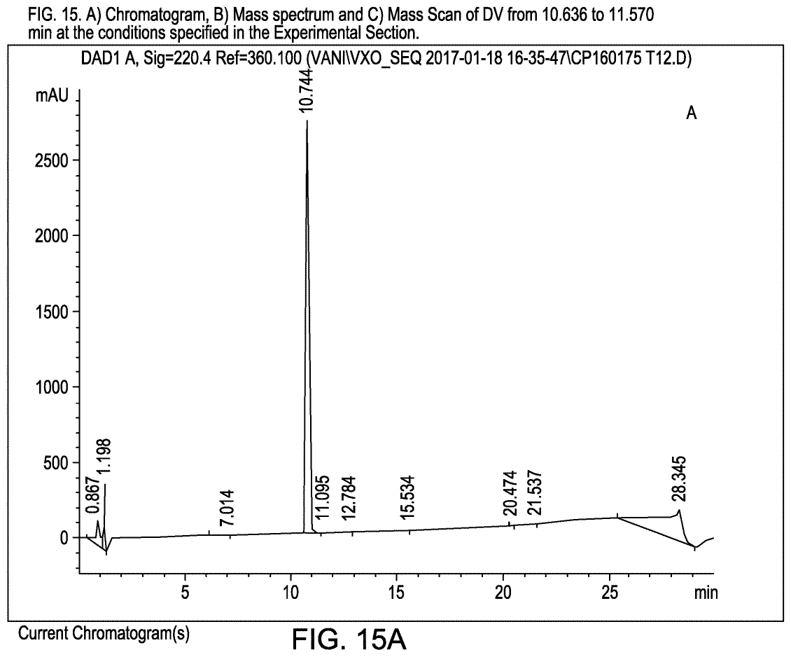

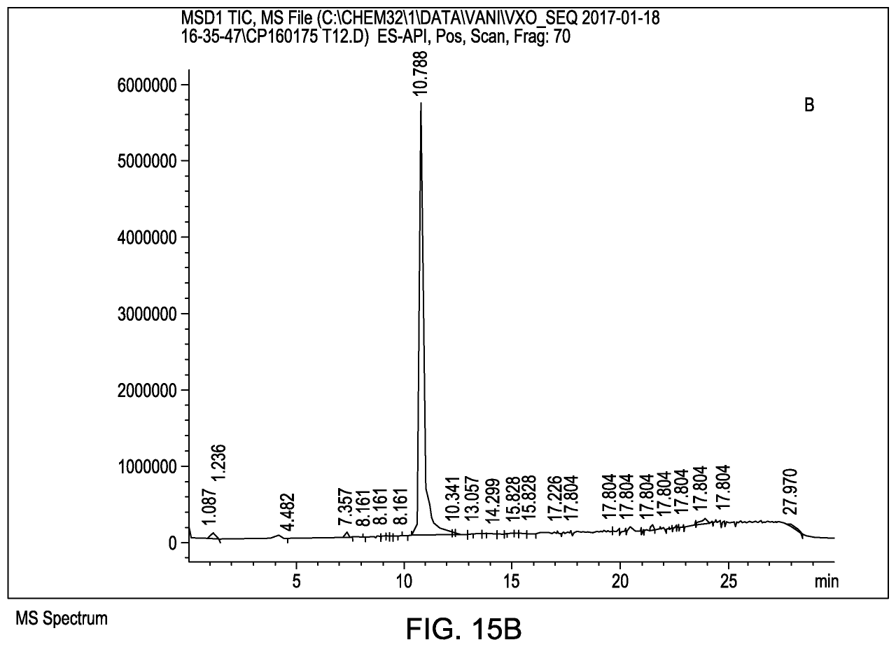

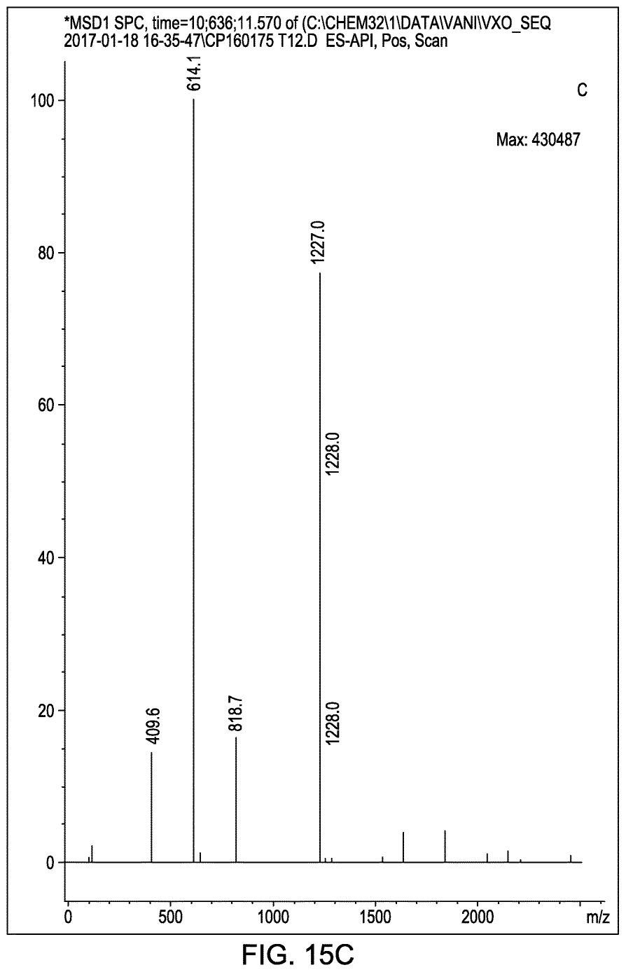

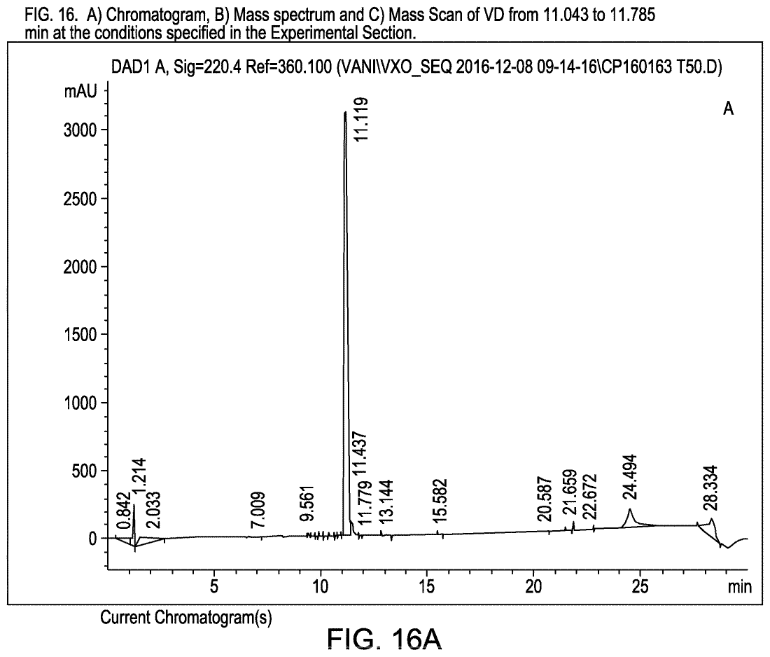

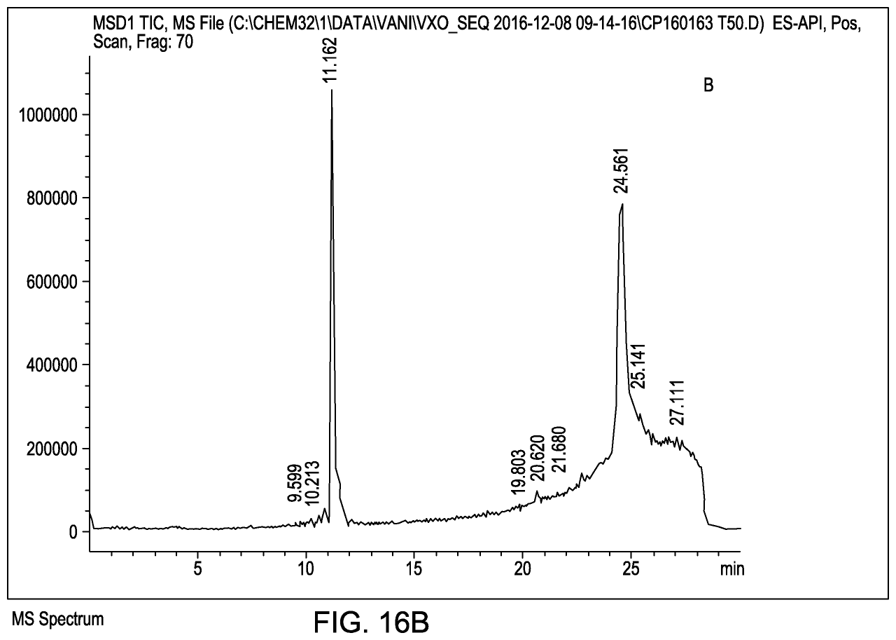

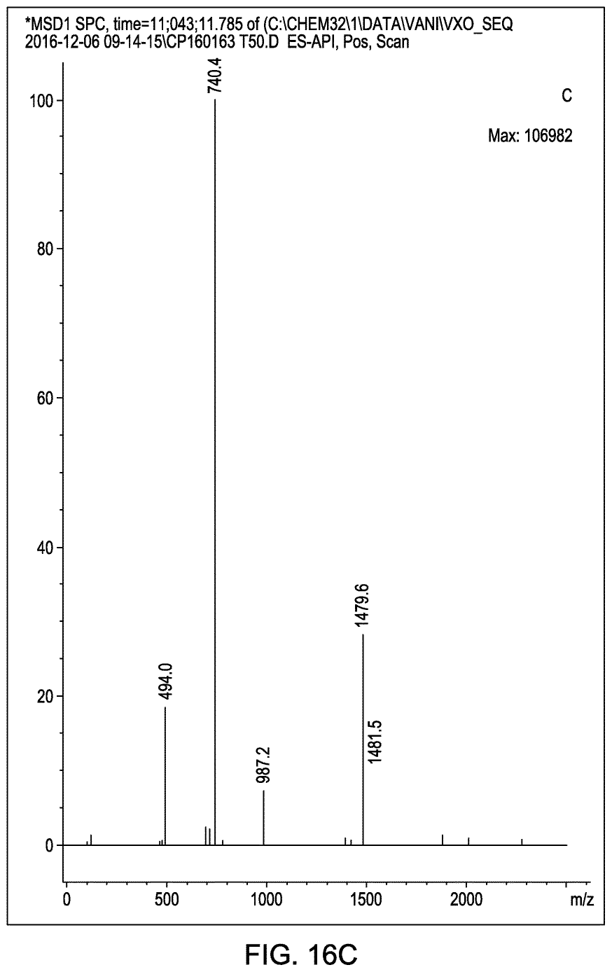

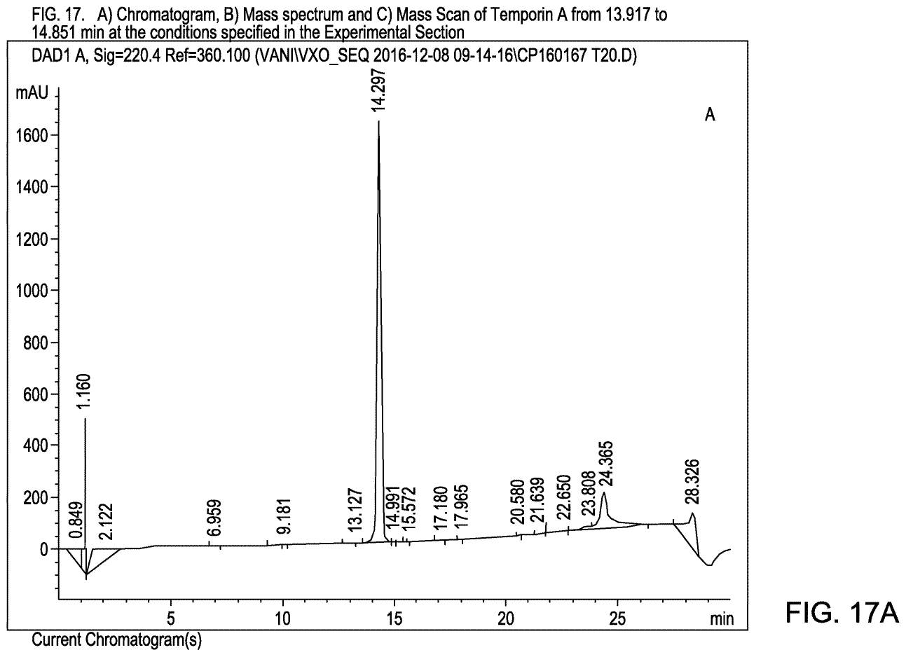

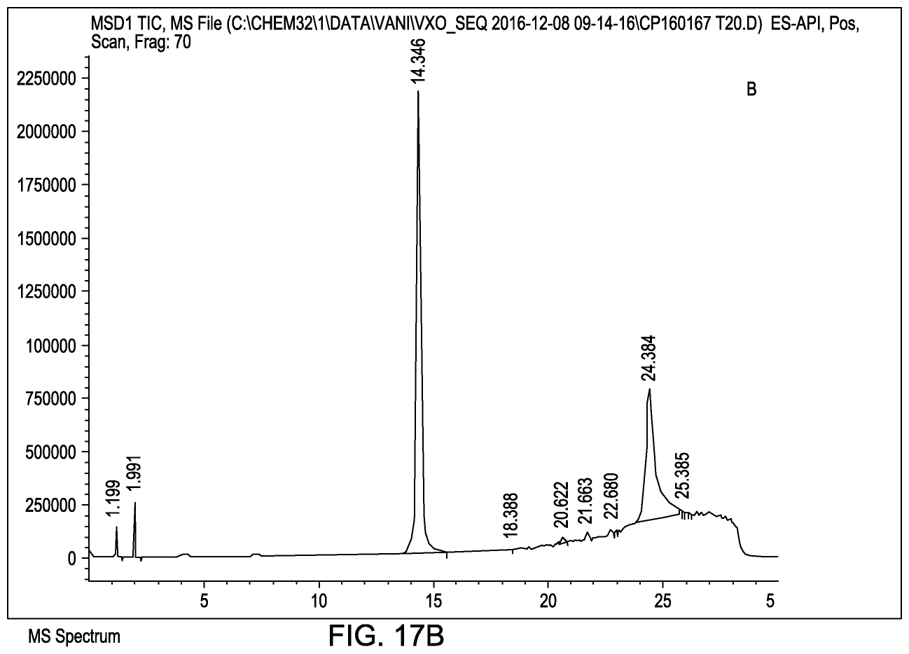

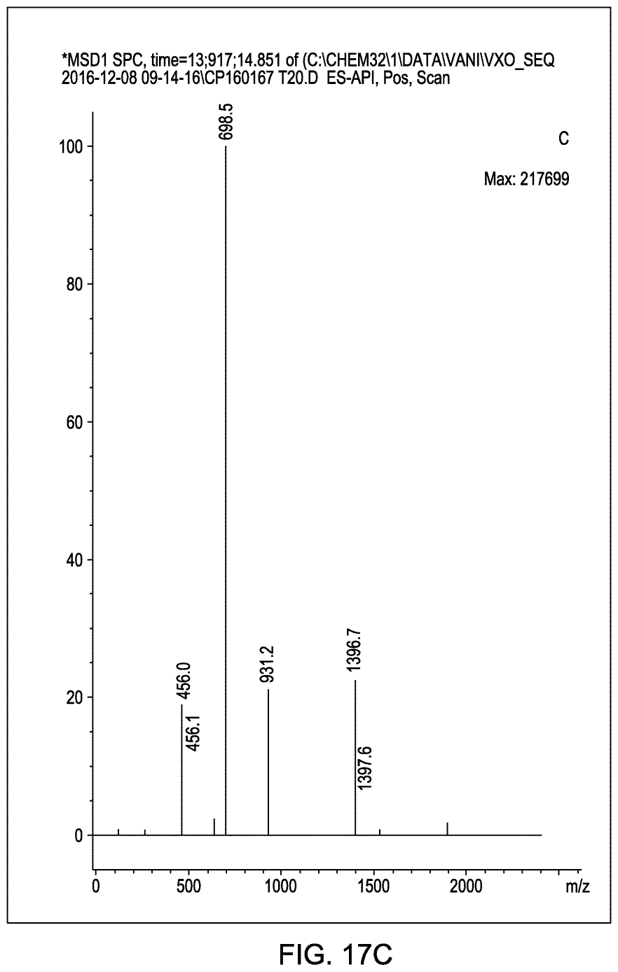

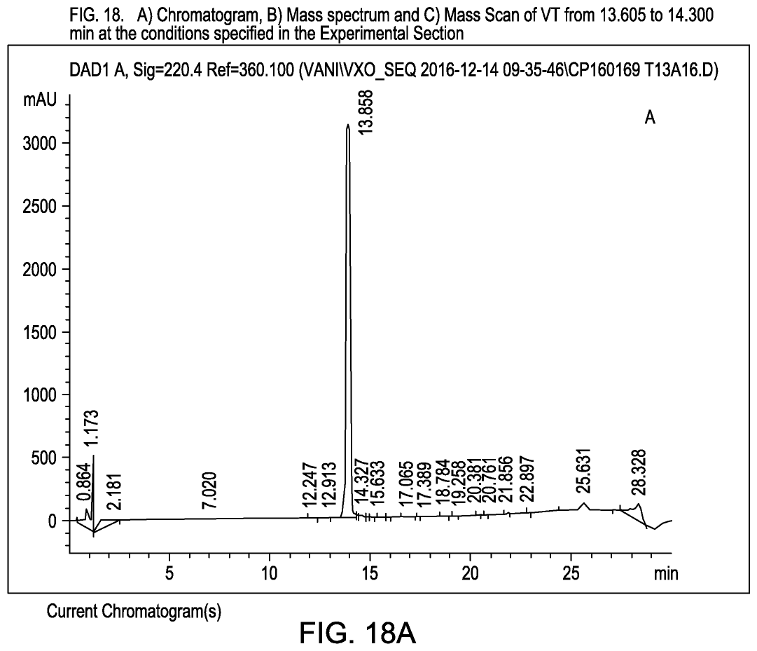

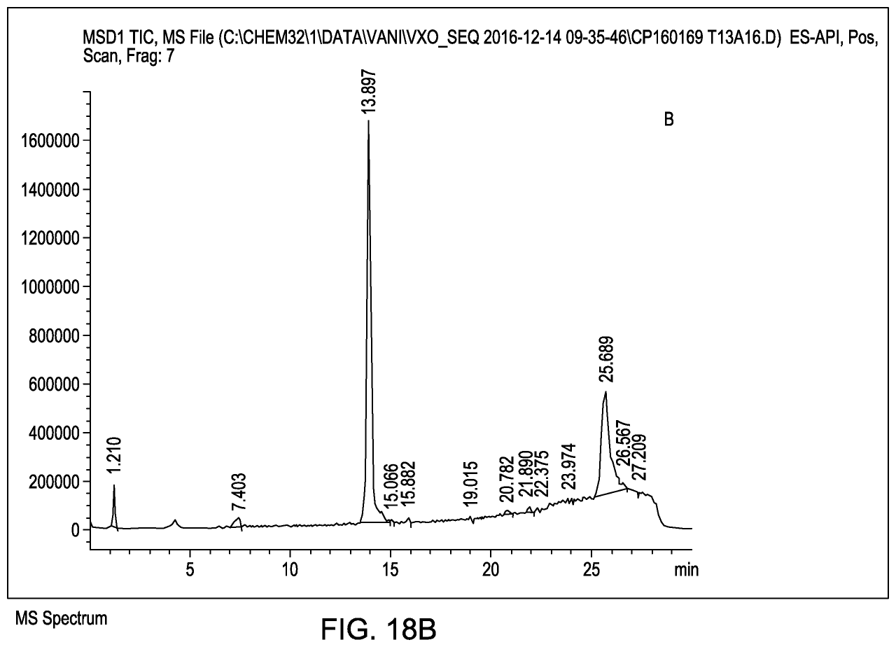

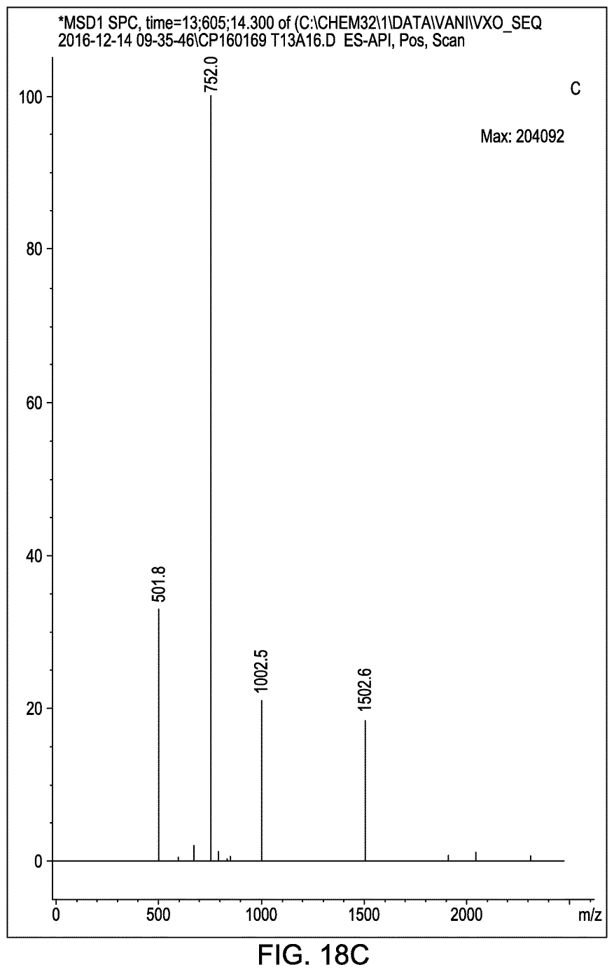

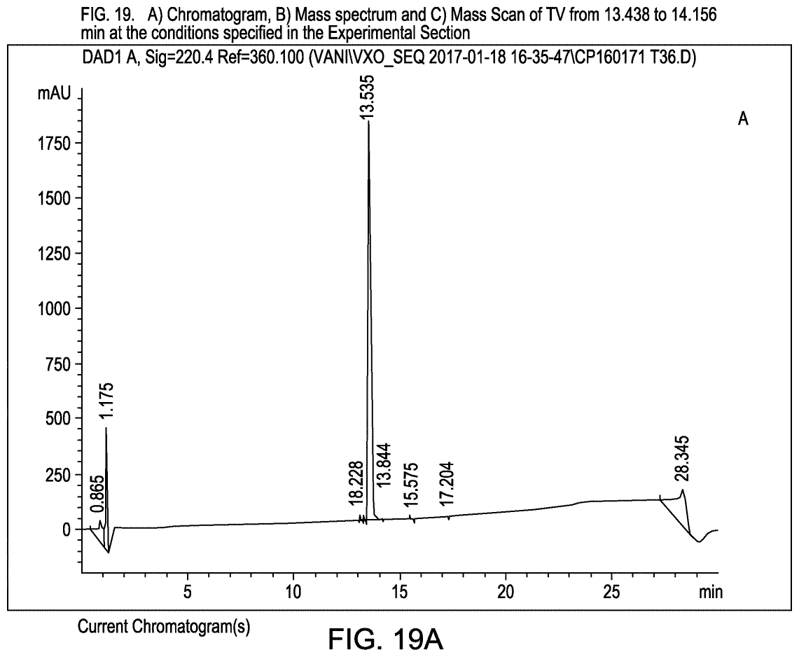

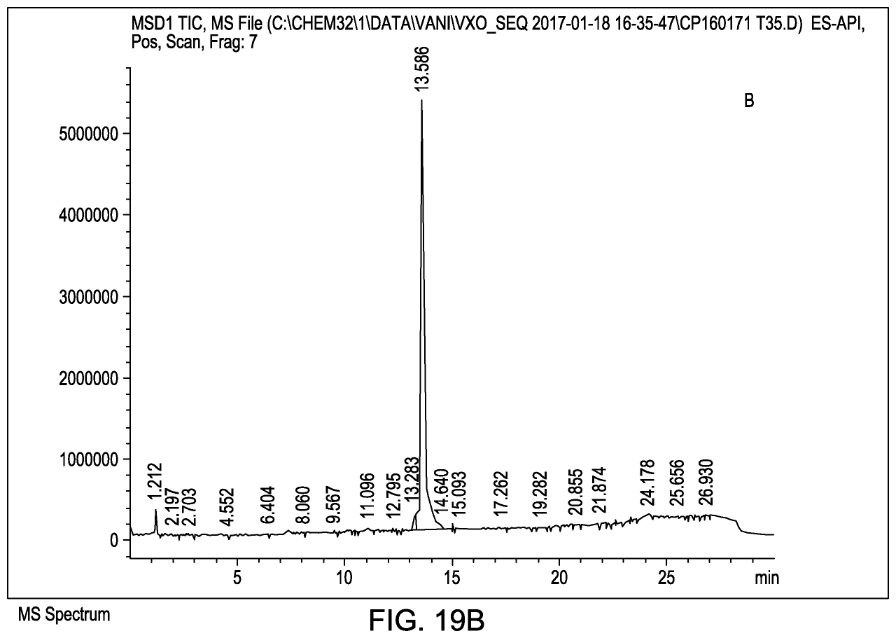

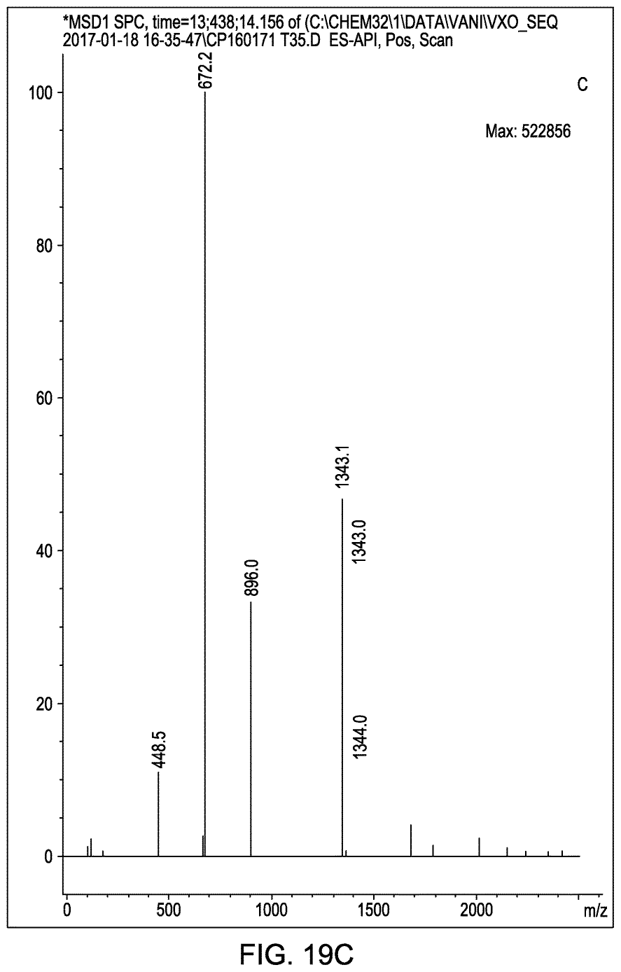

Solid-phase peptide synthesis, purification, and characterization. All peptides were synthesized using solid-phase peptide synthesis (PS3-Sync Technologies) and fluoromethyloxycarbonyl (Fmoc) as described by Torres et al.⁷⁷. Crude peptides were lyophilized and subsequently purified by semi-preparative reverse-phase high-performance liquid chromatography (RP-HPLC) on a Delta Prep 600 (Waters Associates). Selected fractions containing the purified peptides were pooled and lyophilized. The characterization steps were performed by liquid-chromatography electrospray-ionization mass spectrometry (LC/ESI-MS) using a Model 6130 Infinity mass spectrometer coupled to a Model 1260 HPLC system (Agilent), following previously described protocols 71 (FIGS. 8-19). The peptide content was determined by spectrophotometry (Nanodrop™ 2000, Thermo Scientific). To measure the percentage of peptide in the lyophilized sample, the peptides were dissolved in deionized water at a concentration of 1 mg mL⁻¹ and the absorbance was measured at 205 and 260 nm. The percentage of the peptide in the sample was determined by the rate between the expected concentration and the concentration determined experimentally⁷⁸⁻⁸⁰.

### Example 2—Antimicrobial Activity of hAMPs

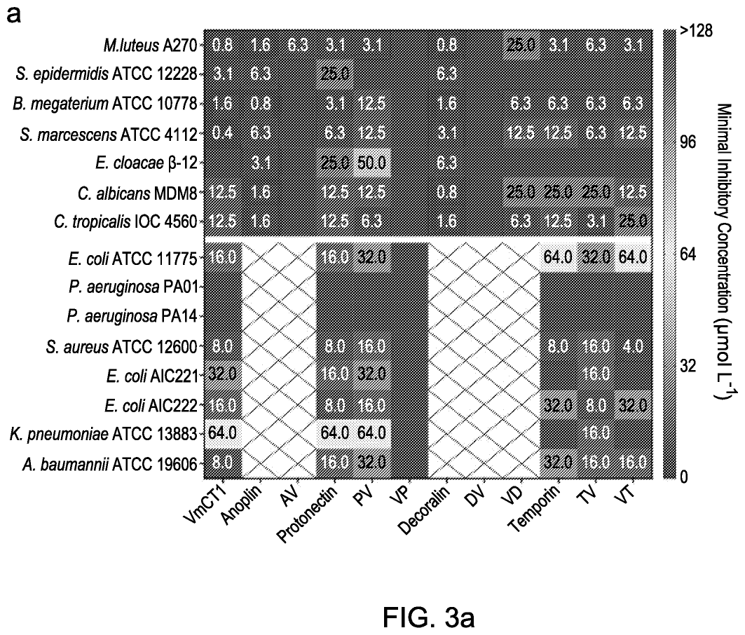

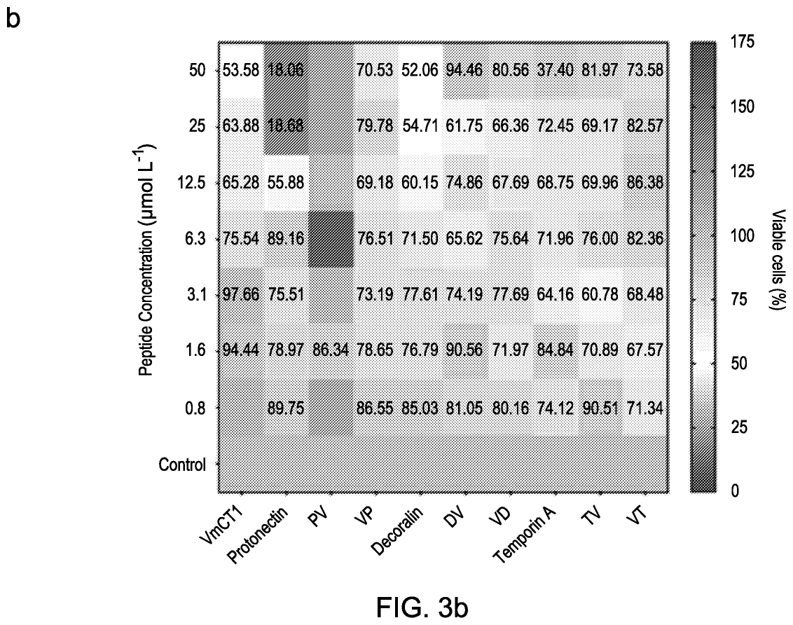

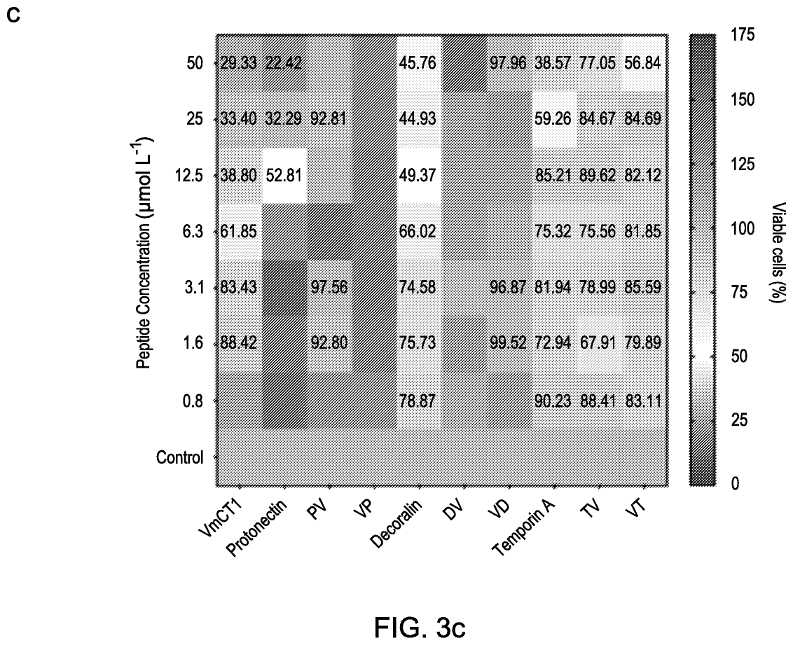

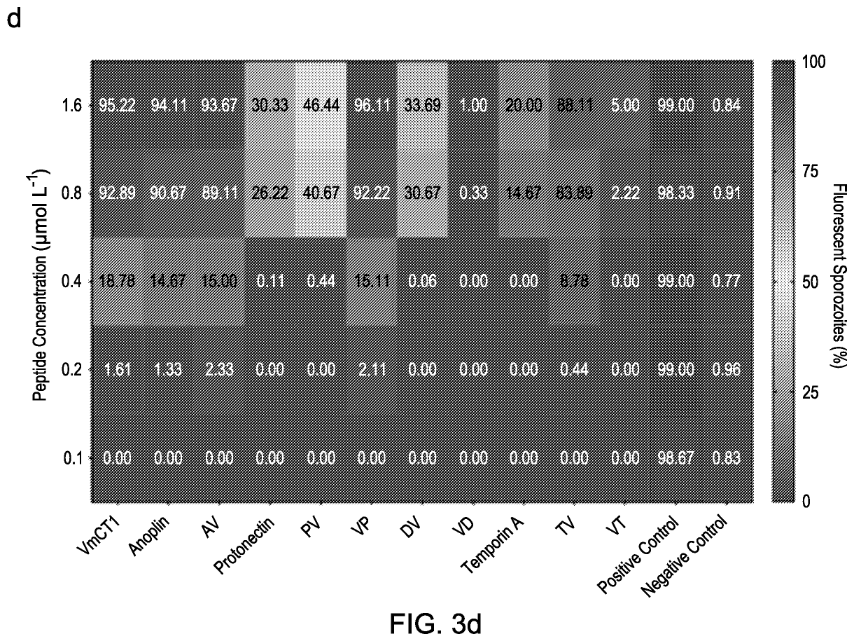

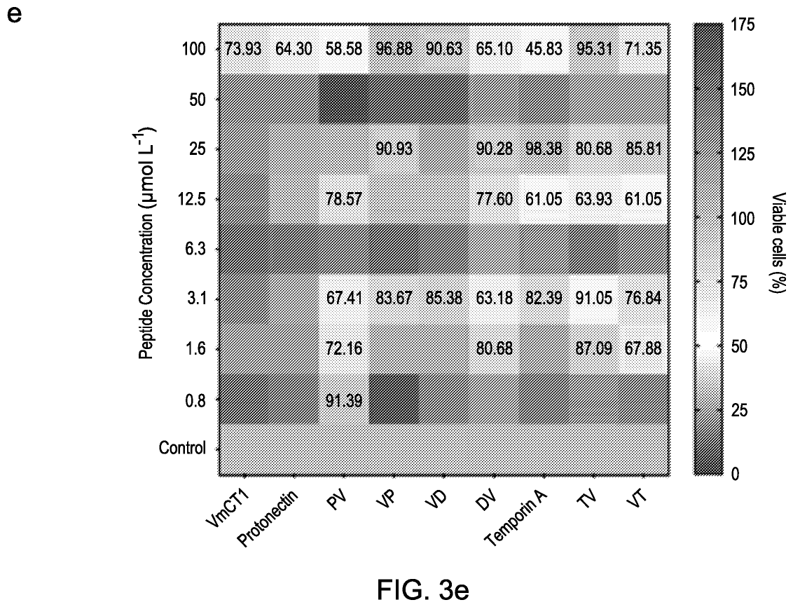

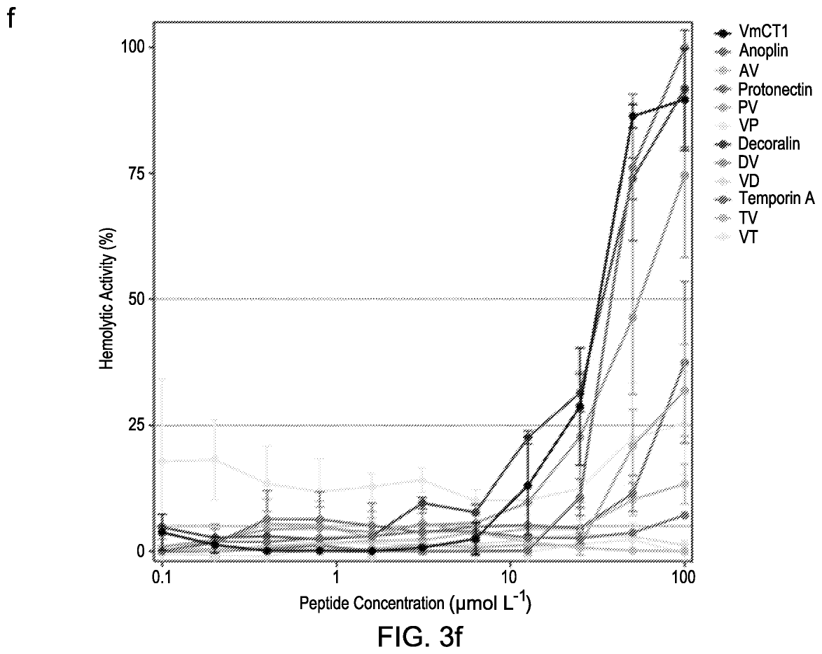

The template peptides (VmCT1, anoplin, protonectin, decoralin, and temporin A) exhibited MICs against bacterial strains ranging from 0.8 to 25 μmol L⁻¹ (FIG. 3a and Tables S1 and S2). Of the seven hybrid peptides tested in this study, those with the highest antibacterial activity (i.e., lowest MICs) were TV and VT: 3.1 to 25 μmol L⁻¹ (FIG. 3a and Tables S1 and S2). TV presented slightly more potent activity against Gram-negative strains (FIG. 3 and Table S1 and S2). The next highest antimicrobial activity was observed with PV (3.1-64 μmol L⁻¹), and VD (6.3-25 μmol L⁻¹). In most cases, the range of antimicrobial activity of the hybrid peptide matched, or came within one dilution, of that of the template AMP (protonecin and temporin A, 3.1-25 μmol L⁻¹). AV and VP, as well as one of the two hybrid peptides derived from decoralin (VD), had lower antimicrobial activities than their corresponding template peptides. The antimicrobial activity (MICs) of AV ranged from 6.3 to 50 μmol L⁻¹, whereas the MICs of anoplin (FIG. 3a and Tables S1 and S2) ranged from 0.8 to 6.3 μmol L⁻¹. No antimicrobial activities were observed with DV. The MIC values of protonectin, PV and VP against P. aeruginosa were 128 μmol L⁻¹ (FIG. 3a and Tables S1 and S2). Anoplin and protonectin were not active against Bacillus subtilis at the range of concentrations tested. AV and VP, as well as the two hybrid peptides derived from decoralin (VD and DV), had lower antimicrobial activities than the corresponding template peptides.

Initially, the antimicrobial activity was determined by liquid growth inhibition assays, as previously described by Pedron et al.⁶². Briefly, Peptone Broth and Potato Dextrose Broth (Invitrogen) were used for antibacterial and antifungal assays, respectively. Two-fold serial dilutions of peptides (0.09-50 μmol L⁻¹) and microorganisms (Serratia marcescens ATCC 4112, Enterobacter cloacae β-12, Micrococcus luteus A270, Bacillus megaterium ATCC 10778, Bacillus subtilis ATCC 6633, Candida albicans MDM8, and Candida tropicalis IOC 4560) were incubated at 37° C. for 18 h (for bacteria) or 24 h (for fungi), and microbial growth was assessed by absorbance measurements at 595 nm. Experiments were performed in three biological repeats.

Method. The minimal inhibitory concentration (MIC) against the ESKAPE pathogens was determined using the broth microdilution method^{81, 82} in Luria-Bertani (LB) medium. The MIC value was defined as the lowest concentration of peptide at which no microbial growth was observed. A. baumannii ATCC19606, E. coli ATCC11775, E. coli AIC221, E. coli AIC222, K. pneumoniae ATCC13883, S. aureus ATCC12600 P. aeruginosa PAO1, and P. aeruginosa PA14 were incubated on agar plates, and after 24 h, three isolated colonies were inoculated to 5 mL of Luria-Bertani (LB) broth and maintained at 37° C. overnight with shaking. Peptides were then added to 96-well polystyrene microtiter plates at concentrations ranging from 0 to 128 μmol L⁻¹, and 5×10⁵ CFU mL⁻¹ of each bacterium was inoculated in each well. The plates were incubated at 37° C. for 24 h. Bacterial growth was measured using a microplate reader at 610 nm. Three independent replicates were performed.

TABLE S1
Biological activity and structural characterization of the peptides.

| | MIC (μmol L⁻¹) | | | | | | | | | | P. gallinaceum |
|---|---|---|---|---|---|---|---|---|---|---|---|
| | M. | S. | B. | S. | E. | C. | C. | MHC | MCF-7 (μmol L⁻¹) | | (% sporozoites |
| | luteus | epidermidis | megaterium | marcescens | cloacae | albicans | tropicalis | (μmol | 4 h | 24 h | at 0.8 |
| Peptide | A270 | ATCC 12228 | ATCC 10778 | ATCC 4112 | β-12 | MDM8 | IOC 4560 | L⁻¹) | LD₅₀ | LD₅₀ | μmol L⁻¹) |
| VmCT1 | 0.8 | 3.1 | 1.6 | 0.4 | >50 | 12.5 | 12.5 | 6.3 | 50 | 12.5 | 93 |
| Anoplin | 1.6 | 6.3 | 0.8 | 6.3 | 3.1 | 1.6 | 1.6 | 50 | NT | NT | 90 |
| AV | 6.3 | >50 | >50 | 50 | >50 | 50 | 50 | >100 | NT | NT | 90 |
| Protonectin | 3.1 | 25 | 3.1 | 6.3 | 25 | 12.5 | 12.5 | 12.5 | 25 | 25 | 26 |
| PV | 3.1 | >50 | 12.5 | 12.5 | >50 | 12.5 | 6.3 | 25 | — | — | 41 |
| VP | >50 | >50 | >50 | >50 | >50 | >50 | >50 | >100 | — | — | 92 |
| Decoralin | 0.8 | 6.3 | 1.6 | 3.1 | 6.3 | 0.8 | 1.6 | 1.6 | — | — | 0 |
| DV | >50 | >50 | >50 | >50 | >50 | >50 | >50 | 0.1 | — | — | 31 |
| VD | 25 | >50 | 6.3 | 12.5 | >50 | 25 | 6.3 | >100 | — | — | 0 |
| Temporin A | 3.1 | >50 | 6.3 | 12.5 | >50 | 25 | 12.5 | 0.2 | 50 | 50 | 15 |
| TV | 6.3 | >50 | 6.3 | 6.3 | >50 | 25 | 3.1 | 0.2 | — | — | 83 |
| VT | 3.1 | >50 | 6.3 | 12.5 | >50 | 12.5 | 25 | 0.2 | — | — | 2 |

MHC value was considered the concentration that there were approximately 0% of hemolytic activity.
NT: the peptide was not tested.

TABLE S2
Antimicrobial activity of the peptides against several ESKAPE pathogens.

| | MIC (μmol L⁻¹) | | | | | | | |
|---|---|---|---|---|---|---|---|---|
| | E. coli | P. aeruginosa | P. aeruginosa | S. aureus | E. coli | E. coli | K. pneumoniae | A. baumannii |
| Peptide | ATCC11775 | PAO1 | PA14 | ATCC12600 | AIC221 | AIC222 | ATCC13883 | ATCC19606 |
| VmCT1 | 16 | 128 | 128 | 8 | 32 | 16 | 64 | 8 |
| Protonectin | 16 | 128 | 128 | 8 | 16 | 8 | 64 | 16 |
| PV | 32 | 128 | 128 | 16 | 32 | 16 | 64 | 32 |
| VP | 128 | 128 | 128 | 128 | 128 | 128 | 128 | 128 |
| Temporin A | 64 | 128 | 128 | 8 | 128 | 32 | 128 | 32 |
| TV | 32 | 128 | 128 | 16 | 16 | 8 | 16 | 16 |
| VT | 64 | 128 | 128 | 4 | 128 | 32 | 128 | 16 |

### Example 3—Antiplasmodial Activity of hAMPs

An assessment was then made of the hybrid peptides for activity against P. gallinaceum, which is the closest model to Plasmodium falciparum infection available with no risk of human infection⁵⁶, using fluorescence microscopy assay, as previously described_{57, 58}. In this assay, fluorescent P. gallinaceum sporozoites are those whose genetic material has been stained by propidium iodide, denoting a damaged membrane (FIG. 3b and Table S1). In this assay, the parent peptides decoralin and temporin A showed no activity. AV, on the other hand, was as active against P. gallinaceum sporozoites as its parent peptides anoplin and VmCT1: 0.8 μmol L⁻¹ of AV and anoplin yielded 90% fluorescent sporozoites, and VmCT1 93% fluorescent sporozoites. VP and TV also displayed antiplasmodial activity: 92% and 83% of the sporozoites were killed by these hybrid peptides, respectively, at 0.8 μmol L⁻¹ (FIG. 3b and Table S1). VD, DV, and VT did not exhibit significant antiplasmodial activity compared to the other molecules designed in this work at the concentration range tested.

Method. The activity of the peptides against Plasmodium gallinaceum sporozoites was determined by fluorescence microscopy, as previously described by Silva et. al.⁸³. Briefly, 9,000 sporozoites were isolated from the glands of infected Aedes aegypti mosquitos and incubated with serially diluted peptides (ranging from 0.1 to 1.6 μmol L⁻¹) at 37° C. for 1 h. A PBS solution and the surfactant digitonin^{84, 85} were used as negative and positive controls, respectively. Propidium iodide was used to stain dead cells. Assays were executed using fluorescence microscopy. Three biological repeats each with three different mosquito batches (n=9) were used.

### Example 4—Activity of hAMPs on Healthy and Cancer Human Cell Lines

In comparison with healthy cell membranes, cancerous cell membranes typically present higher levels of anionic molecules, such as phosphatidylserines, glycoproteins with net negative charge, and glycosaminoglycans, favoring electrostatic interactions between cationic peptides and the cell membrane⁵⁹⁻⁶¹. Since the design principles used here led to peptides with activity against negatively charged cell membranes, we assessed the activity of the hybrid peptides (except for anoplin and AV) against MCF-7 human breast cancer cells after 4 and 24 h of incubation (FIG. 3c and Table S1).

VD, unlike the other hybrid peptides, had selective activity against MCF-7 human breast cancer cells at 25 μmol L⁻¹. However, VD showed decreased activity compared to its template peptides VmCT1 and decoralin (50 μmol L⁻¹). Within 4 h of incubation with MCF-7, protonectin and temporin A reduced the counts of viable MCF-7 cells (LD₅₀: 25 μmol L⁻¹ and 50 μmol L⁻¹, respectively). After 24 h of incubation, protonectin and temporin A exhibited the same LD₅₀ (LD₅₀: 25 μmol L⁻¹ and 50 μmol L⁻¹, respectively). As a control, our designed peptides were also tested against MCF-10A healthy human breast epithelial cells (FIG. 3d). After 24 h of incubation, we did not observe peptide-meditated cytotoxicity towards MCF-10A cells (FIG. 3e).

The anticancer activity assays were performed using MCF-10A human breast epithelial cells and MCF-7 mammary cancer cells as previously described by Pedron et al. 86. Briefly, the day before treatment, the cells were incubated at 37° C. and 5% CO₂ for 24 h. The cells were treated with peptides ranging from 12.5 to 100 μmol L⁻¹ (MCF10-A cells) and 0.8 to 100 μmol L⁻¹ (MCF-7 cells) for 4 h and 24 h at 37° C., and cell viability was measured using 3-(4,5-Dimethylthiazol-2-yl)-2,5-diphenyltetrazolium bromide (MTT) assays 87. Three independent replicates were performed in each case.

### Example 5—Hemolytic Activity of hAMPs

Hemolytic activity is a key measure of potential toxicity or safety for human cells. Hemolytic activity, here defined as the concentration of peptide needed to kill 10% of red blood cells, was observed with the parent peptides decoralin and temporin, which were hemolytic at 1.6 μmol L⁻¹ and 0.2 μmol L⁻¹, respectively. Anoplin was hemolytic at 50 μmol L⁻¹ (FIG. 3f and Table S1). DV was significantly hemolytic at low concentrations (0.1 μmol L⁻¹), and VT and TV presented hemolytic activity at 0.2 μmol L⁻¹ (FIG. 3f and Table S1). The hybrid peptides having the lowest hemolytic activity were AV and VP (100 μmol L⁻¹) along with VD, for which no hemolytic activity was detected in our assay (FIG. 3f and Table S1). The markedly improved hemolytic profile of VP (hemolytic at 100 μmol L⁻¹) compared to the parent peptide protonectin (hemolytic at 12.5 μmol L⁻¹) or to PV (hemolytic at 25 μmol L⁻¹) suggests that peptide hybridization can produce derivatives with improved safety profiles.

Method. Assays against freshly collected human erythrocytes were performed following protocols described by Pedron et al. 62. Briefly, peptide aliquots at concentrations ranging from 0.1 to 100 μmol L−1 were incubated with a suspension of erythrocytes at room temperature for 1 h. The samples were then centrifuged, and the absorbance of the supernatant was measured at 405 nm. The MHC (maximal non-hemolytic concentration) was determined as the maximal concentration at which 100% of the erythrocytes were viable. The surfactant SDS and PBS buffer were used as positive88, 89 and negative controls, respectively. Sodium heparin was used as an anticoagulant when collecting human blood. Three biological repeats were performed.

### Example 6—Relationship of Structural Characteristics to Peptide Activity

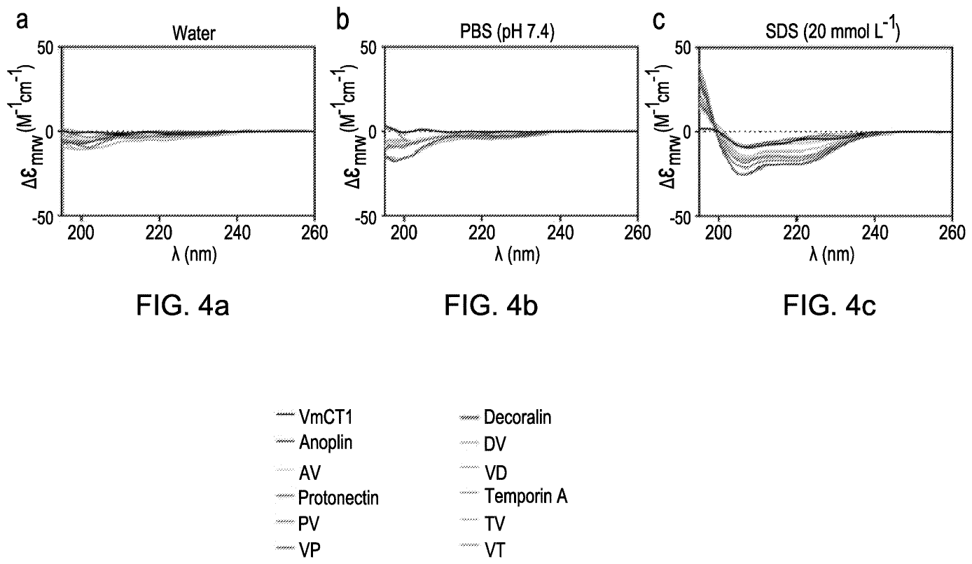

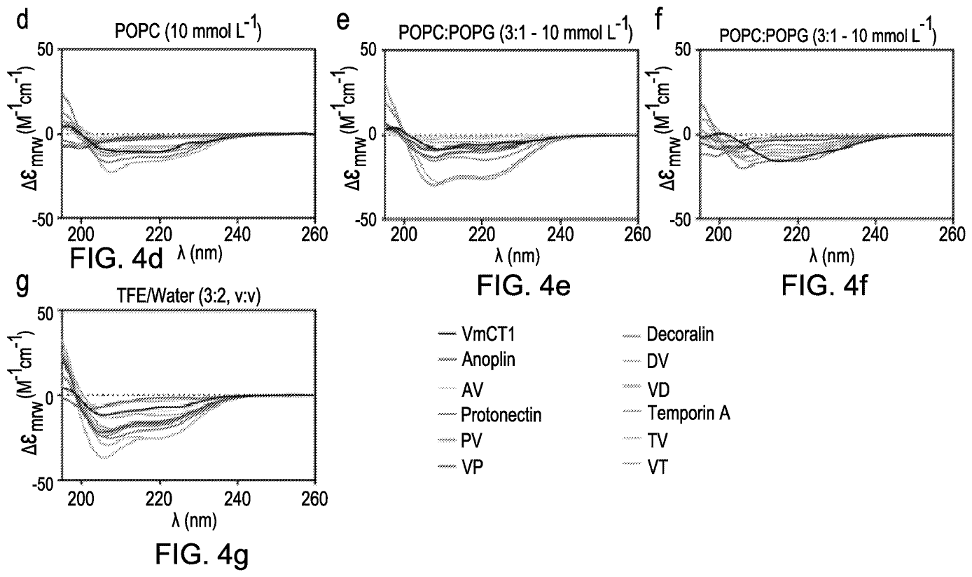

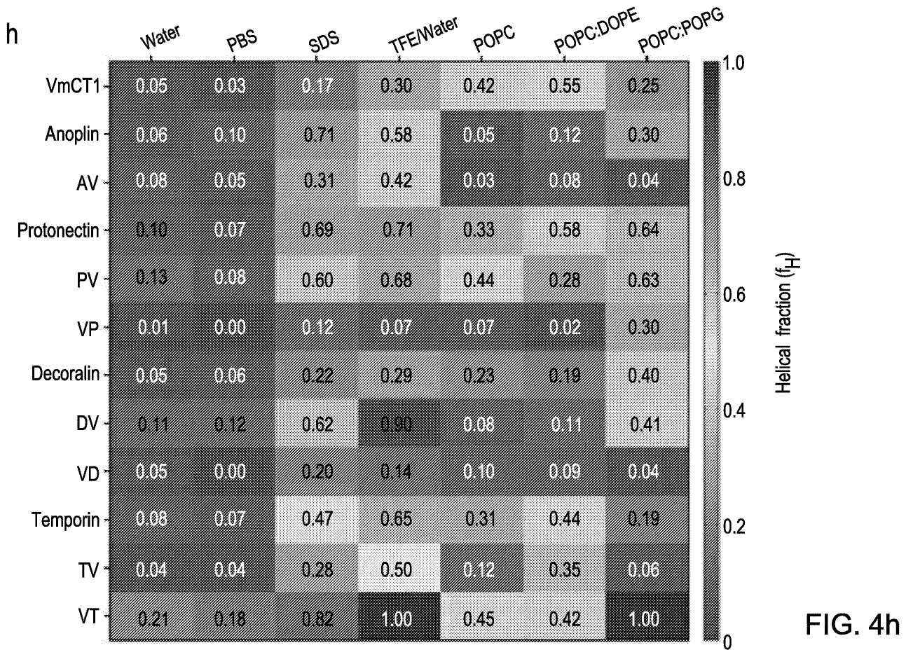

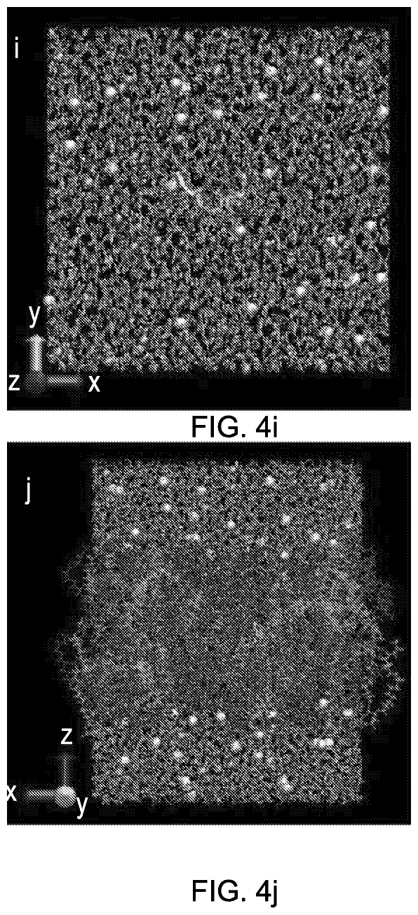

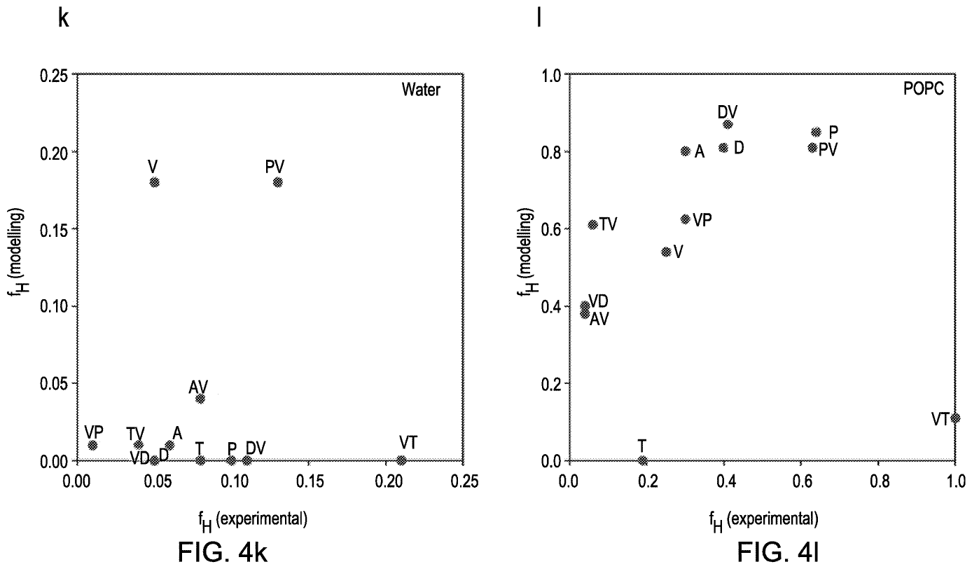

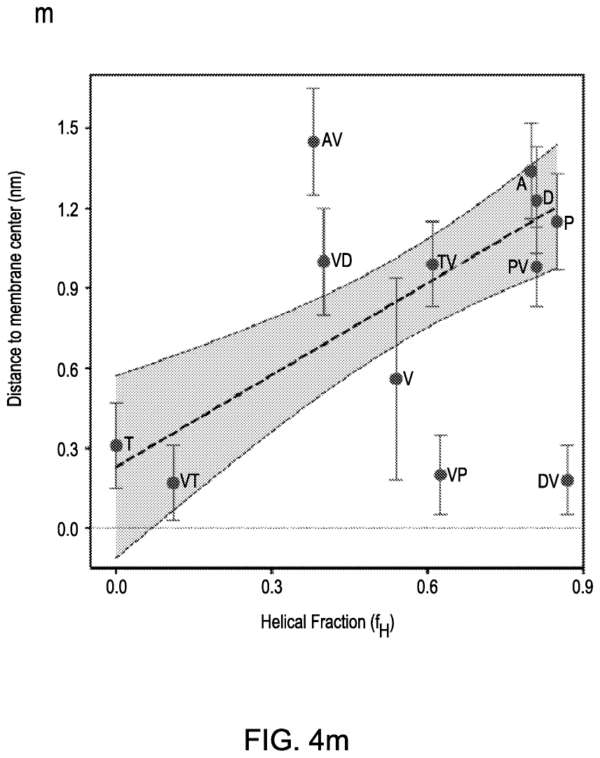

To assess whether we could link the observed bioactivities to the structural characteristics of the hybrid peptides, we evaluated their secondary structure by circular dichroism (CD; FIGS. 4a-g). Anoplin is an α-helical AMP isolated from Anoplius samariensis wasp venom. This peptide is composed of 10 amino acid residues and has an amidated carboxyl-terminus⁴⁰. AV, which has Gly residues in positions 1 and 12, presented a lower net charge (+2) than anoplin itself and had a lower hydrophobic moment than VmCT1 or anoplin (Table 1).

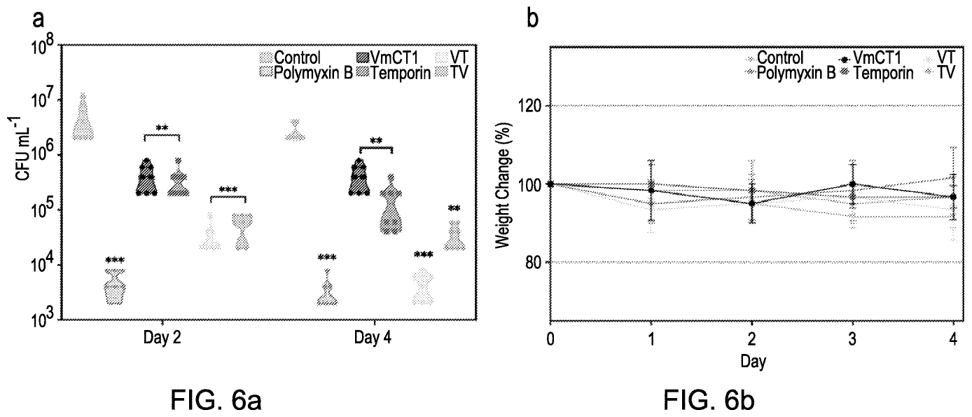

Pedron et. al. reported that Lys-substitutions of VmCT1 analogs increased the net positive charge of this family of peptides and were important for their increased antimicrobial activity⁶². Munk et. al. synthesized 19 anoplin analogs and found that changes in net positive charge and hydrophobicity-related properties influenced activity against erythrocytes and microorganisms⁶³. Therefore, we attribute the lower antimicrobial activity of AV to its lower net positive charge value compared to its parent molecules. The helical content of AV was lower than that of anoplin for all the media analyzed and higher than the helical content of VmCT1 in SDS (f_{H}: 0.31 and 0.17, respectively) and TFE/water solution (f_{H}: 0.42 and 0.30, respectively) (FIG. 6h and Table S3).

Protonectin, an AMP isolated from Agelaia pallipes wasp venom, is composed of 12 amino acid residues and presented hemolytic activity at 12.5 μmol L⁻¹, but shows potent antimicrobial activity against bacteria⁵⁴. VP was designed with one additional Gly residue placed at position 8.

The CD assays (FIGS. 4a-g) suggested that the active hybrid peptides derived from VmCT1 and protonectin presented high (>60%) helical content. VP exhibited low helical content values in all the media tested, whereas PV presented higher helical content than VmCT1 in all media used, with helical fraction values higher than 0.5, e.g., SDS (fir: 0.60) POPC:POPG (f_{H}: 0.63) and TFE/water solution (f_{H}: 0.68) (FIGS. 4c, 4e, and 4g, respectively, and FIG. 4h and Table S3) and higher helical content than protonectin in POPC (f_{H}: 0.44 and 0.33, respectively) (FIGS. 4d and 4h and Table S3).

Protonectin displayed homology with polybia-CP, though it differs at position 11, where protonectin has a Gly residue and polybia-CP contains a Ser residue. Torres et al. described an Alanine-scan screening of polybia-CP and verified that modifications within the hydrophobic face of the peptide decreased its antimicrobial activity, revealing that its hydrophobic face was crucial for antimicrobial activity₂₇. These observations are consistent with our results for PV, which incorporated protonectin's hydrophobic face (FIG. 2) displaying increased antimicrobial activity than VP (FIG. 3). Additionally, VP exhibited high antiplasmodial activity (92% of killed sporozoites) at 0.8 μmol L⁻¹ (FIG. 3b and Table S1). In contrast, PV presented lower antiplasmodial activity at the same concentration killing only 41% of the sporozoites (FIG. 3b and Table S1). The antiplasmodial activity observed for this peptide confirms that its helical content is less important to its antiplasmodial activity than to its antimicrobial and cytotoxic activities (FIGS. 3a-c and Table S1). These results are in line with previous observations by Torres et al., who showed that the anti-Plasmodium activity of small cationic decoralin analogs did not depend on their α-helical structure 57. The authors showed that decoralin's N-terminal extremity motif was crucial for its antiplasmodial activity. Decoralin, an AMP isolated from the venom of the Oreumenes decoratus wasp and containing 11 amino acid residues and an amidated C-terminal extremity, tends to adopt a helical structure and has broad-spectrum antimicrobial activity and low hemolytic activity at 100 μmol L^{-1 42}.

Peptide VD, which had two Gly residues as spacers in positions 3 and 10, presented low helical content in all media tested. On the contrary, DV exhibited higher helical content than VmCT1 in all media used (FIGS. 4a-h and Table S3). No effect was found between a low helical content and hemolytic or antimicrobial activities. DV, which also had two additional Gly residues at positions 12 and 13, did not exhibit antimicrobial activity at the range of concentrations tested and was significantly hemolytic even at low concentrations (0.1 μmol L⁻¹). Torres et al. 64 similarly described Leu-substituted decoralin analogs, in which the peptide with a Leu-substitution at position 8 presented lower helical content in TFE/water solution (f_{H}: 0.24) than decoralin and had increased antimicrobial activity and lower hemolytic activity with a minimal hemolytic concentration (MHC) value of 50 μmol L⁻¹ (Table S1), showing that changes in conformational tendencies affected both antimicrobial and hemolytic activities. Thus, helical structure was not found to be a determinant of antimicrobial activity for hybrid peptides derived from VmCT1 or decoralin.

The antiplasmodial activity of decoralin analogs was previously described by Torres et al.⁵⁷. Although decoralin itself did not present antiplasmodial activity, rationally designed synthetic decoralin analogs had antiplasmodial activity at 60 μmol L⁻¹, i.e., a concentration higher than that of the hybrid peptides designed in the present study. Like the native decoralin, VD and DV did not present antiplasmodial activity (FIG. 3b and Table S1).

The temporins are a group of peptides with 10 to 13 amino acid residues in their sequence^{48, 65}. Temporin A exhibits activity against Gram-positive and Gram-negative bacteria and low hemolytic activity⁴⁸, which made it a promising candidate for hybridization with VmCT1. VT and TV presented higher helical content values than VmCT1. VT also presented higher helical content than temporin A, but TV had lower helical content values than temporin A in all media tested (FIG. 4h and Table S3), the increased observed helicity of the hybrids did not lead to increased bioactivity compared to the templates (FIG. 4h).

TABLE S3
Helical fraction of the wild-type and analogs in seven different
media calculated by using Lifson-Roig helix-coil theory.⁷⁷

| | Helical fraction (f_{H}) | | | | | | |
|---|---|---|---|---|---|---|---|
| Peptide | Water | PBS | SDS | TFE/Water | POPC | POPC:DOPE | POPC:POPG |
| VmCT1 | 0.05 | 0.03 | 0.17 | 0.30 | 0.42 | 0.55 | 0.25 |
| Anoplin | 0.06 | 0.10 | 0.71 | 0.58 | 0.05 | 0.12 | 0.30 |
| AV | 0.08 | 0.05 | 0.31 | 0.42 | 0.03 | 0.08 | 0.04 |
| Protonectin | 0.10 | 0.07 | 0.69 | 0.71 | 0.33 | 0.58 | 0.64 |
| PV | 0.13 | 0.08 | 0.60 | 0.68 | 0.44 | 0.28 | 0.63 |
| VP | 0.01 | 0 | 0.12 | 0.07 | 0.07 | 0.02 | 0.30 |
| Decoralin | 0.05 | 0.06 | 0.22 | 0.29 | 0.23 | 0.19 | 0.40 |
| DV | 0.11 | 0.12 | 0.62 | 0.90 | 0.08 | 0.11 | 0.41 |
| VD | 0.05 | 0 | 0.20 | 0.14 | 0.10 | 0.09 | 0.04 |
| Temporin A | 0.08 | 0.07 | 0.47 | 0.65 | 0.31 | 0.44 | 0.19 |
| TV | 0.04 | 0.04 | 0.28 | 0.50 | 0.12 | 0.35 | 0.06 |
| VT | 0.21 | 0.18 | 0.82 | 1.00 | 0.45 | 0.42 | 1.00 |

Phosphate buffer saline (PBS, 10 mmol L⁻¹, pH 7.4), sodium dodecyl sulfate (SDS 20 mmol L⁻¹) in water, 2,2,2-trifluoroethanol (TFE) in water (3:2, v:v), palmitoyloleoylphosphatidylcholine (POPC; 10 mmol L⁻¹), palmitoyloleoylphosphatidylcholine:dioleylphosphatidylethanolamine (POPC:DOPE; 3:1, mol:mol, 10 mmol L⁻¹) and palmitoyloleoylphosphatidylcholine:palmitoyloleoylphosphatidylglycerol (POPC:POPG; 3:1, mol:mol, 10 mmol L⁻¹).

Method. Circular dichroism (CD) assays were performed with a Spectropolarimeter Jasco Mod. J-815 (JascoCorp). CD spectra were obtained in Far-UV (195-260 nm), and analyzed in the following solutions: water, 2,2,2-trifluoroethanol (TFE) in water (3:2, v:v), phosphate buffer saline (PBS, 10 mmol L⁻¹, pH 7.4), sodium dodecyl sulfate (SDS 20 mmol L⁻¹) in water, palmitoyloleoylphosphatidylcholine (POPC; 10 mmol L⁻¹), palmitoyloleoylphosphatidylcholine:dioleylphosphatidylethanolamine (POPC:DOPE; 3:1, mol:mol, 10 mmol L⁻¹) and palmitoyloleoylphosphatidylcholine:palmitoyloleoylphosphatidylglycerol (POPC:POPG; 3:1, mol:mol, 10 mmol L⁻¹) at a peptide concentration of 50 μmol L⁻¹. These experiments were performed as described in detail by Pedron et al.⁶².

### Example 7—Molecular Dynamics (MD) Simulations

To gather more information on the structure of the hAMPs and their parent peptides, we performed molecular dynamics (MD) studies (FIGS. 4i-j). First, we calculated the average helical fraction over the last 1,000 ns of the simulation trajectory according to the STRIDE algorithm⁶⁶ implemented in the Visual Molecular Dynamics (VMD) software⁶⁷. Each residue at each time frame is assigned one out of eight secondary structure types defined by the STRIDE algorithm depending on their phi (Φ) and psi (Ψ) dihedral angles. The residue is considered to form helical structure if it adopts any of the three secondary structure types, α-helix, 3-₁₀ helix, or α-helix.

The helical structures of AMP peptides are stabilized because of the presence of POPC:POPG membrane in MD simulation (FIGS. 4k-1), which qualitatively agree with the results obtained by CD measurements in POPC:POPG vesicles (FIG. 4h and Table S3). The qualitative comparison is encouraging; however, the peptides are relatively over-destabilized in aqueous solution and are over-stabilized (except for T and VT) in lipid membrane compared to the experimental values. There are several possible reasons for explaining the deviations, including (1) the limited sampling time; (2) the choice of specific protein force fields; (3) the definition of the helical fraction in simulation is different from that of the experimental measurements.

Next, the influence of the helical fraction on the membrane penetration depth of the parent peptides and the hAMPs was investigated (FIG. 4m). Nine out of twelve AMPs exhibited positive correlation between their helical fraction and the distance to membrane center where AMP with higher helical fraction is more surface active and stays closer to the water-membrane interface (FIG. 4m). We hypothesize that this is caused by the amphipathic nature, with hydrophobic residues and hydrophilic residues distributed orderly on each half of the two cylindrical faces from the helical wheel projection (FIG. 2), what maximizes contact with lipid and water environments, respectively.

It was also found that the three AMPs (AV, VP and DV), which do not have a correlation between their helical fraction values and the distance to membrane center (FIG. 4m), all share a similar sequence feature; that is, they each have more than three residues with low hydrophobicity (Gly or Ala), but not polar. Specifically, for the case of VP and DV in our simulations, they form stable helical structures, and the prevalence of hydrophobic residues allow whole peptide to preferentially interact the hydrophobic membrane center. Interestingly, VP and DV, are the two peptides that have the weakest antimicrobial activity against all bacteria strains (MIC≥128 μmol L−1, FIG. 3a and Tables S1 and S2). Additionally, the fact that AV is more surface active (closer to the water-lipid interface) than VP may be explained by the more balanced distribution of hydrophobic and hydrophilic residues on AV's sequence. Conversely to VP, AV has one more polar non-charged residue (Asn) on the hydrophilic face and does not have residues with large hydrophobic side chains, such as the Trp or Phe bulked aromatic side chains found on the hydrophobic side of VP's sequence (FIG. 2). Altogether, the simulations results show that in most cases the design assumptions considering helical structure as a main factor for hybridization were correct and led to hAMPs with high antimicrobial activity.

Method. Molecular dynamics (MD) simulations were performed using GROMACS package, 2020.6 version on Nvidia A100 GPU workstations. Peptides and phospholipids are modeled using the CHARMM36m force field⁹⁰. Tip3p water model⁹¹ is used for modeling water solvent. Cutoff distances for both van der Waal and columbic potential are 1.2 nm. Particle mesh Ewald (PME) summation method is applied for considering long-range electrostatic interactions⁹².

The initial configurations of the twelve AMP-membrane systems were prepared using the CHARMM-GUI membrane builder⁹³. The initial structure of VmCT1 (V), Protonectin (P) and Decoralin (D) are taken from the protein data bank (PDB code: lt54⁹⁴, 6n68⁹⁵, 2n9a⁹⁶). The initial configurations of Anoplin (A) and TemporinA (T) are obtained by replacing certain residues from protein data bank structure (PDB code: 2mjr⁹⁷ and 6gil⁹⁸, respectively) using the SCWRL program⁹⁹. The initial configurations for the rest 7 hAMP are obtained by replacing residues from VmCT1 structure (PDB code: lt54) using the same program. Heterogeneous membrane bilayer consists of POPC and POPG (ratio 3:1), with each leaflet containing 64 lipids. Sodium cations and chloride anions were used to neutralize the system using a salt concentration of 0.15M. Simulation box size is around 6.5×6.5×8.2 nm³, with periodic boundary conditions applied in all three directions. First, steepest descent method was applied for energy minimization of initial configuration, which was subsequently equilibrated by isothermal-isobaric (NPT) simulation for a few nanoseconds at 1 bar using the Berendsen barostat¹⁰⁰. For production stage, the pressure is maintained constant at 1 bar using the Parrinello-Rahman barostat¹⁰¹. The temperature was maintained at 300K using the Nos é-Hoover thermostat¹⁰². A time step of 2 fs is used, and the simulation trajectories were saved every 100 ps. Bonds with hydrogen atoms are constrained using the LINCS algorithm¹⁰³. The total simulation time for each system is about 1.5 μs. All analyses in this study are performed on the last 1 μs of the production trajectories.

For the twelve AMP-water systems, the simulation box size is set up in such a way that the distance between any atom from the AMP peptide to the boundary of a cubic simulation box is at least 2.4 nm to prevent any molecule from interacting with its periodic image during the simulation. The MD simulations of 12 AMP-water systems are performed under the same conditions with those for AMP-membrane systems and last about 1.5 μs.

### Example 8—hAMPs Resistance to Proteolytic Degradation

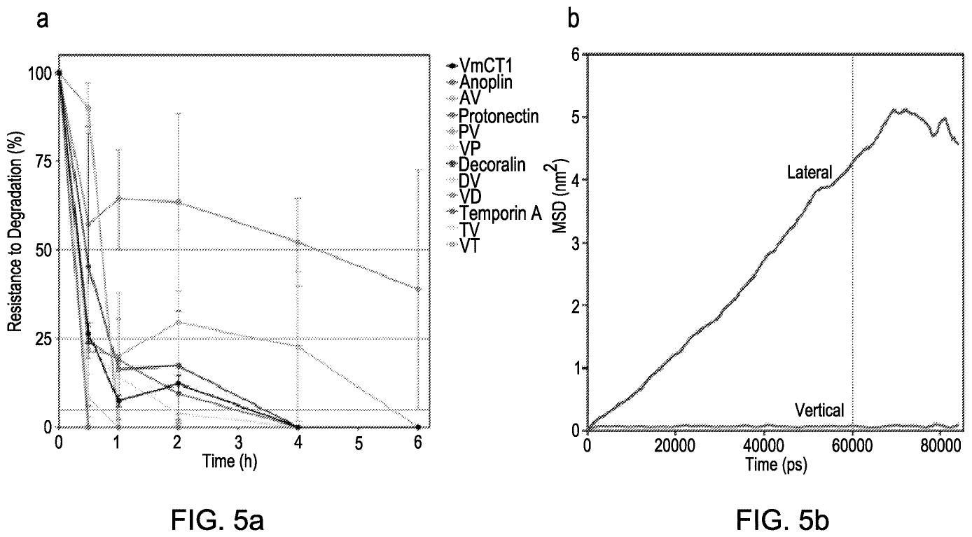

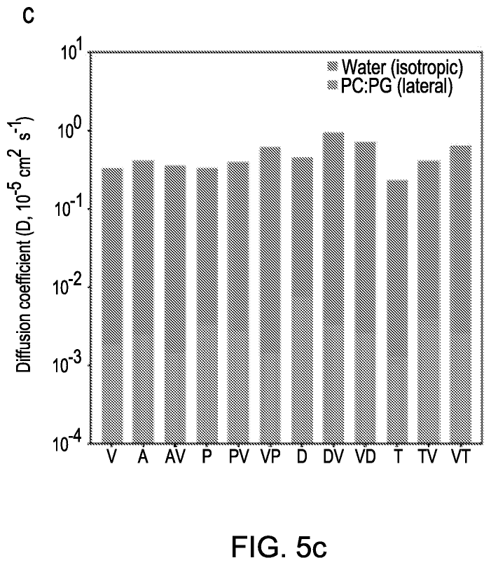

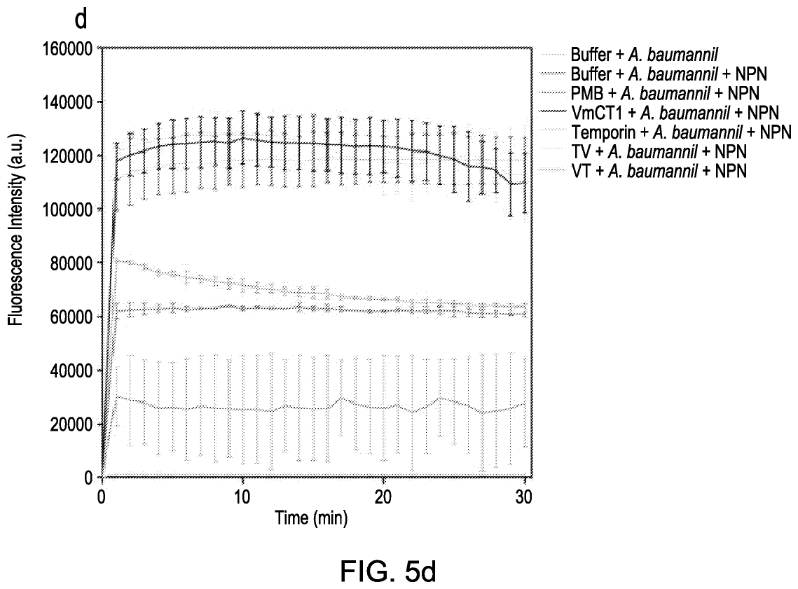

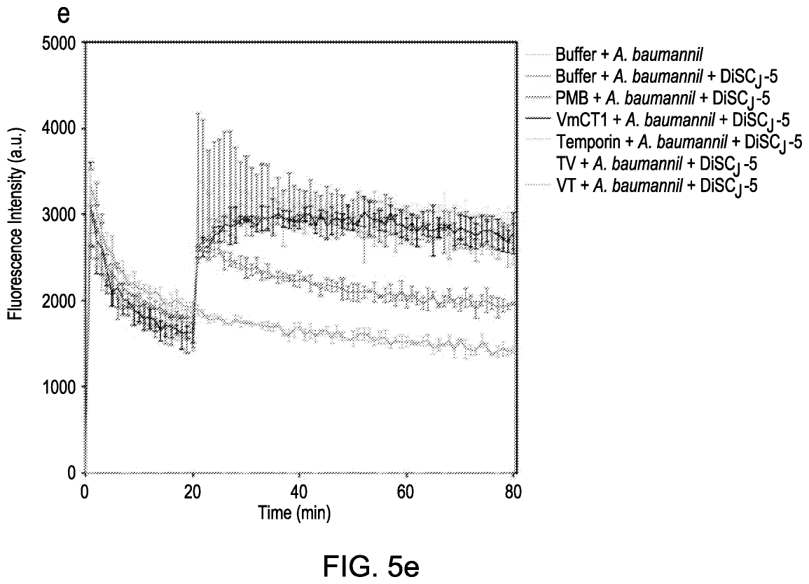

The resistance to proteolytic degradation of the hybrid peptides was assessed by exposing them for 6 h to serum peptidases in a fetal bovine serum solution^{68, 69} (FIG. 5a and Table S4). The peptide degradation kinetics were calculated by integrating the peak area of the peak in relation to the peptide at the beginning of the experiment (time=0) and comparing it to the integrated peak area relative to the peptides after exposure to the enzymes at time points: 0.5, 1, 2, 4, and 6 h (FIG. 5a and Table S4). VmCT1 was quickly degraded, with approximately 20% of peptide remaining after 30 min of exposure to serum proteases and less than 10% after 1 h (FIG. 5a and Table S4). Anoplin was slightly more stable: less than 50% of peptide remained after 30 min of exposure to peptidases. However, AV was resistant to degradation against proteases, with almost 40% peptide content remaining after 4 h of exposure (FIG. 5a and Table S4). Protonectin was quickly degraded (FIG. 5a and Table S4), whereas PV resisted for 30 min (~90% of peptide remaining); however, it was totally degraded after 1 h of exposure. VP was almost entirely degraded after 30 min (22% of peptide remaining). Temporin A, TV, and VT were totally degraded within 30 min after the start of the experiment (FIG. 5a and Table S4). D-amino acid substitutions have been used as alternatives to prevent the degradation of AMPs. For example, Qiu et. al. described a protonectin analog with a D-Lys residue in position 10 as an alternative to protonectin; this analog is not degraded and has higher antimicrobial activity against bacteria and fungi than the original peptide. In addition, the authors showed that D-protonectin was stable in the presence of trypsin, chymotrypsin, and human serum, whereas D-Lys-protonectin showed stability only against trypsin⁷⁰.

The most resistant peptide among our synthesized analogs was VD, the decoralin-derived hAMP, which has a Trp residue in position 6. VD resisted degradation (~50% of peptide remaining) for 6 h (FIG. 5a and Table S4). Torres et al. reported the importance of position 6 in the decoralin sequence to the resistance to peptidases⁷¹. The authors verified that substituting position 6 (Ile) by a Phe residue created analogs with higher resistance to degradation⁷¹.

TABLE S4
Resistance to degradation studies. Peptides were exposed to fetal bovine
serum enzymes for 6 h and the remaining peptides was calculated based on liquid
chromatography coupled to mass spectrometry experiments.

| Peptide | 30 min | 1 h | 2 h | 4 h | 6 h |
|---|---|---|---|---|---|
| VmCT1 | ~20% | <10% | <10% | — | — |
| Anoplin | ~20% | ~20% | ~10% | — | — |
| AV | ~40% | ~40% | ~40% | ~40% | — |
| Protonectin | — | — | — | — | — |
| PV | ~ 90% | — | — | — | — |
| VP | ~20% | <20% | <10% | — | — |
| Decoralin | ~70% | <30% | <30% | — | — |
| DV | ~50% | ~50% | ~50% | ~50% | ~50% |
| VD | ~10% | — | — | — | — |
| Temporin A | — | — | — | — | — |
| TV | — | — | — | — | — |
| VT | — | — | — | — | — |

Method. Membrane permeabilization was assessed for two of the naturally occurring peptides (VmCT1 and temporin A) and two of the hybrids [VmCT1-temporin A (VT) and temporin A-VmCT1 (TV)] at their MIC by using the N-phenyl-1-naphthylamine (NPN) uptake assay as previously described^{104, 105}. Briefly, A. baumannii ATCC19606 cells were grown to an optical density at 600 nm (OD₆₀₀) of 0.4, centrifuged (10,000 rpm at 4° C. to 10 minutes), washed, and resuspended in buffer (5 mmol L⁻¹ HEPES, 5 mmol L⁻¹ glucose, pH 7.4). Four μL of NPN solution (0.5 mmol L⁻¹; working concentration of 10 μmol L⁻¹, after dilutions) was added to 100 μL of the bacterial solution in 96-well plates. The background fluorescence was then determined at λₑₓ=350 nm and λₑₓ=420 nm. The peptides resuspended in water (100 μL at a concentration of 1.2 μmol L⁻¹) were added to each 96-well plate. The fluorescence was determined as a function of time until no further increase in fluorescence was observed (45 min).

### Example 9—Mechanism of Action of hAMPs

To understand whether the parent peptides and the hAMPs designed would preferably target biological membranes (similar to most known AMPs), we calculated the diffusion coefficient of all peptides in the presence and absence of the membrane. According to the Stokes-Einstein equation, the self-diffusion coefficient is equal to the slope of the mean square displacement (MSD) of all atoms on the peptide sequence⁷²,

$${D}_{d} = \frac{1}{2 d} {lim}_{t \to ∞} \frac{d}{dt} MSD , \qquad ( 1 )$$

where D is the diffusion coefficient and subscript d is the number of degrees of freedom. d=2 and 3 for lateral (in the plane of the membrane) diffusion and isotropic diffusion, respectively. The mean square displacement MSD= [symbol] |r(Δt)−rₒ|² [symbol] was calculated as the average displacement of all atom coordinates r on the peptide during the time interval Δt from a reference point rₒ.

For instance, VmCT1 displays anisotropic diffusion on the PC:PG membranes, where they are diffusive only in the XY plane but display sub-diffusive behavior in the vertical (Z) direction (FIG. 4i-j). The MSD values for other AMPs are similar to VmCT1 but are not shown here. Furthermore, the isotropic diffusion of AMP in aqueous solution is about a 100-fold larger than that of lateral diffusion on lipid membrane, indicating that the motion of AMP is strongly suppressed due to their interaction with lipid head and tail on the bilayer membrane (FIG. 5c).

To assess the disruptive and permeabilizing effect of the most active hAMPs (TV and VT) and their parent peptides VmCT1 and temporin A on the outer membrane of A. baumannii bacteria, we used the probe 1-(N-phenylamino)-naphthalene (NPN), as previously described^{73, 74}. The peptides VmCT1, temporin A, and TV increased fluorescence compared to the control, indicating the permeabilization of A. baumannii cell membranes. On the contrary, the hybrid peptide VT did not lead to increased fluorescence compared to the control (FIG. 5d).

Membrane depolarization constitutes another mechanism of antibacterial activity. To study whether the hybrid peptides derived from temporin A depolarized cytoplasmic membranes, we used the probe 3,3′-dipropylthiadicarbocyanine iodide [DiSC₃(5)]^{73, 74}. The hybrid peptides tested, VT and TV, depolarized the cytoplasmic membrane of A. baumannii, presenting increased fluorescence compared to the controls (FIG. 5e).

Method. The cytoplasmatic membrane depolarization activity of two of the naturally occurring peptides (VmCT1 and temporin A) and two of the hybrids (VT and TV) was determined at their MIC by performing the membrane potential-sensitive dye DiSC₃(5), as reported previously^{105, 106.} A. baumannii ATCC19606 cells were grown at 37° C. with agitation to an optical density of 600 nm (OD₆₀₀) of 0.5, centrifuged, washed twice with buffer (5 mmol L⁻¹ HEPES, 20 mmol L⁻¹ glucose, pH 7.2), and resuspended to an OD₆₀₀ of 0.05 in the same buffer but containing 0.1 mol L⁻¹ KCl. Cells (100 μL) were incubated for 15 minutes with 20 nmol L⁻¹ of DiSC₃(5) until a stable reduction in fluorescence was observed, indicating the incorporation of the DiSC₃(5) into the bacterial membrane. After adding the peptides (100 μL solution at 1.2 μmol L⁻¹), membrane depolarization was determined by the change observed in the fluorescence emission intensity of the dye at λₑₓ=622 nm and λₑₓ=670 nm.

### Example 10—Skin Abscess Infection Mouse Model and Histopathological Assays

The most potent peptides described in this study (FIG. 6a), VmCT1, temporin A, VT, and TV, were evaluated for their anti-infective activity in a skin infection mouse model^{27, 74, 75} against A. baumannii, which is responsible for nosocomial and secondary infections with high death rates⁷⁶. The skin infection was made by a superficial abrasion on the back of the mouse, which injures the stratum corneum and the upper layer of the epidermis. A. baumannii (10⁷ CFU) was inoculated at the scarification site. As a proxy for toxicity, we monitored the weight and visible changes, such as redness, lethargy, morbidity, and loss of fur, over the course of the experiment. Two days post-treatment, TV and VT showed increased anti-infective activity when compared to the parent peptides (VmCT1 and temporin A) and results that are statistically comparable to those obtained with the conventional antibiotic polymyxin B (PMB). Four days after the treatment, both hybrid peptides TV demonstrated significant anti-infective activity comparable to PMB, reducing the load of A. baumannii cells in the infected area by two orders of magnitude (FIG. 6a) and VT presented the same effect as the two parent peptides. Peptide treatment did not lead to significant changes in weight (FIG. 6b) or other visible side effects.

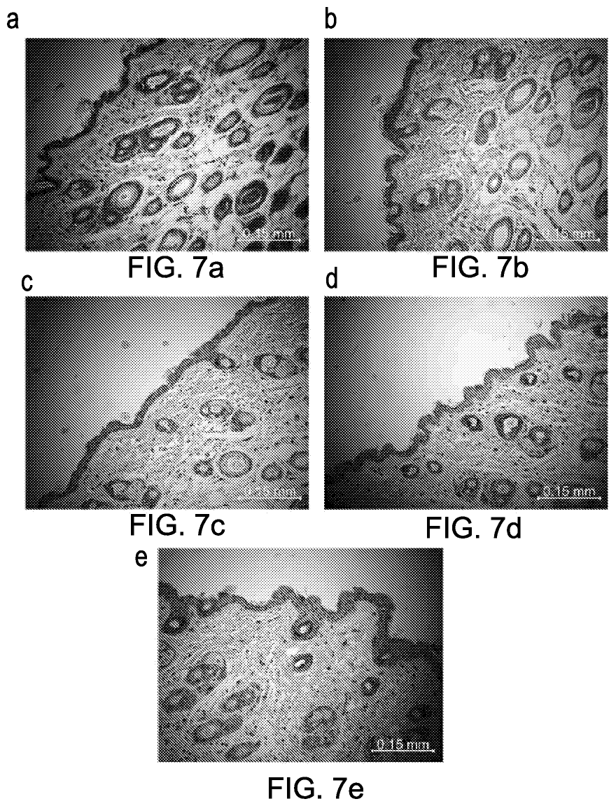

Next, the most active hybrid peptide (TV) and its parent VmCT1 were selected to assess their histopathological effect on skin tissue. First, we performed the skin abscess infection mouse model and euthanized the mice and collected their tissue 24 h after infection and treatment. Hematoxylin and eosin (H&E) staining were used to stain the nuclei and extracellular matrix and cytoplasm, respectively, followed by optical microscopy (FIG. 7). Neither TV nor VmCT1 changed the tissue's morphology compared to the control polymyxin B and the infected untreated or uninfected samples. Thus, we further confirmed that the peptides studied here did not display toxicity.

In this study, a series of hybrid peptides were designed by combining the hydrophobic and hydrophilic faces of the amphipathic helical structure of the venom peptide VmCT1 with parts of other active AMPs (i.e., anoplin, protonectin, decoralin, and temporin A) of different sizes and physicochemical and structural properties. The results show that hybridization of the amphipathic regions of AMPs represent as a tool for generating new peptides and that some of these may exhibit broad-spectrum activity, anti-infective activity, and lower cytotoxicity for human cells, compared to the original peptides. The results also indicate that peptide hybridization is useful for analyzing whether hydrophilic and hydrophobic faces contribute to the overall biological activity of the hybrid peptides and potentially offer a guide for constructing AMPs with improved activity profiles.

The hybrid peptides were also tested for their resistance to enzymatic degradation and mechanisms of action. Among the hybrid peptides, VD exhibited the greatest resistance to proteolytic degradation. The investigation of the mechanism of action showed that the hybrid peptides TV and VT, similar to their parent peptides VmCT1 and temporin A, either disrupted or depolarized the bacterial membrane and revealed their multifunctional mechanism of action. By rearranging the amphipathic regions of peptides it is possible to create hybrids that overcome activity restrictions and instability in the presence of proteases, which currently limit the application of AMPs in the clinic.

In addition to investigating the antibacterial activity of the hybrid peptides, their interactions with eukaryotic cells was investigated. Several small cationic peptides (AV, VP, and TV) exhibited antiplasmodial activity; our study revealed that the helical content was not an important feature for this activity. The approach also enabled the generation of several hAMPs with minimal hemolytic activity (e.g., AV, VP, and VD). None of the designed peptides exhibited cytotoxic activity against MCF-10A healthy human breast epithelial cells (FIG. 3e).

Finally, two of the hybrid peptides were tested in a mouse model of skin infection. In this skin scarification mouse model, VT and TV had anti-infective activity more potent than that of either parent peptides, VmCT1 and temporin A.

Methods. Stability assays were performed following the protocol described by Torres et al.⁷¹. Briefly, a peptide solution was added to GIBCO fetal bovine serum diluted to 25% in water and kept at 37° C. The degradation kinetics were monitored by liquid chromatography and the percentage of peptide remaining was calculated by integrating the peptide peak area. The experiments were performed as three independent replicates, and aliquots were taken at 0, 0.5, 1, 2, 4, and 6 h.

Skin abscess infection mouse model. The anti-infective activity of the peptides VmCT1, temporin A, VT, and TV against A. baumannii ATCC19606 was evaluated as described by Torres et al.⁸². Bacteria were grown in tryptic soy broth medium to an optical density of 0.5 at 600 nm. Cells were washed twice with sterile phosphate buffered saline (pH 7.4, 13,000 rpm for 1 min) and resuspended to a final concentration of 1×10⁷ CFU/20 μL. To generate the skin infection, female CD-1 mice (6 weeks-old) were first anesthetized with isoflurane and their backs were shaved. A superficial linear abrasion was made with a needle on the shaved area to injure only the stratum corneum and the upper layer of the epidermis. An aliquot of 20 μL suspension containing 1×10⁷ CFU of A. baumannii was then inoculated over the wound area with a pipette tip. The next day, the infected area was inoculated with the peptides (VmCT1, temporin A, VT, and TV), which were administered onto the abscess at their MIC. The animals were euthanized, and the scarified skin area removed 2- and 4-days post-infection. The excised area was homogenized using a bead beater for 20 min (25 Hz) and serially diluted for CFU quantification. Two independent experiments were carried out with 3 mice per group in each condition.

Skin tissue histopathological analysis. The histopathological effect of the parent peptide VmCT1 and hAMP TV (the most active hybrid peptide in vivo) was assessed by hematoxylin and eosin (H&E) staining of the skin tissue followed by microscopy analysis. The fur on the back of the mice was removed and the mice were infected and treated as described in the Skin abscess infection mouse model section. After 24 h, mice were euthanized for tissue collection. Samples were pre-fixed in individual cassettes, then transferred to a container filled with 70% EtOH for slicing and staining. Next, the samples were placed on glass slides for microscopy analysis. We collected the tissue from three mice per condition analyzed.

In sum, the present inventors have demonstrated that the hybridization of amphipathic portions of AMPs is an effective design strategy for generating peptides with broad-spectrum activity at sub-micromolar concentrations and minimal hemolytic and cytotoxic activities toward human cells. The hybridization of portions of known peptides was heretofore an underexplored molecular engineering tool, and the present invention represents a novel strategy for the design of bioactive peptides.

### REFERENCES

The following references may be relevant to the present disclosure:
1 Tacconelli, E. et al. Discovery, research, and development of new antibiotics: the WHO priority list of antibiotic-resistant bacteria and tuberculosis. Lancet Infect Dis 18, 318-327 (2018).
2. Murray, C. J. et al. Global burden of bacterial antimicrobial resistance in 2019: a systematic analysis. The Lancet 399, 629-655 (2022).
3. World Health Organization. WHO World Malaria Report 2020. Malaria report 2020 (2020).
4 Mahlapuu, M., Håkansson, J., Ringstad, L. & Björn, C. Antimicrobial Peptides: An Emerging Category of Therapeutic Agents. Front Cell Infect Microbiol 6, 1-12 (2016).
5 Marqus, S., Pirogova, E. & Piva, T. J. Evaluation of the use of therapeutic peptides for cancer treatment. J Biomed Sci 24, 1-15 (2017).
6. de la Fuente-Nunez, C., Torres, M. D., Mojica, F. J. & Lu, T. K. Next-generation precision antimicrobials: towards personalized treatment of infectious diseases. Curr Opin Microbiol 37, 95-102 (2017).
7. Cesaro, A. et al. Synthetic Antibiotic Derived from Sequences Encrypted in a Protein from Human Plasma. ACS Nano 16, 1880-1895 (2022).
8. Torres, M. D. T., Melo, M. C. R., Crescenzi, O., Notomista, E. & de la Fuente-Nunez, C. Mining for encrypted peptide antibiotics in the human proteome. Nat Biomed Eng 6, 67-75 (2022).
9. Vinhote, J. F. C. et al. Trypanocidal activity of mastoparan from Polybia paulista wasp venom by interaction with TcGAPDH. Toxicon 137, 168-172 (2017).
10. Chu, H. L. et al. Novel antimicrobial peptides with high anticancer activity and selectivity. PLOS One 10, 1-14 (2015).
11. Der Torossian Torres, M. & de la Fuente-Nunez, C. Reprogramming biological peptides to combat infectious diseases. Chemical Communications 55, 15020-15032 (2019).
12. Torres, M. D. T. & de la Fuente-Nunez, C. Toward computer-made artificial antibiotics. Curr Opin Microbiol 51, 30-38 (2019).
13. Torres, M. D. T., Cao, J., Franco, O. L., Lu, T. K. & de la Fuente-Nunez, C. Synthetic Biology and Computer-Based Frameworks for Antimicrobial Peptide Discovery. ACS Nano 15, 2143-2164 (2021).
14. Torres, M. D. T., Sothiselvam, S., Lu, T. K. & de la Fuente-Nunez, C. Peptide Design Principles for Antimicrobial Applications. J Mol Biol (2019) doi: 10.1016/j.jmb.2018.12.015.
15. Jenssen, H., Hamill, P. & Hancock, R. E. W. Peptide antimicrobial agents. Clin Microbiol Rev 19, 491-511 (2006).
16. Durnaœ, B. et al. Candidacidal activity of selected ceragenins and human cathelicidin LL-37 in experimental settings mimicking infection sites. PLOS One 11, 1-21 (2016).
17. De Breij, A. et al. The antimicrobial peptide SAAP-148 combats drug-resistant bacteria and biofilms. Sci Transl Med 10, (2018).
18. E. Greber, K. & Dawgul, M. Antimicrobial Peptides Under Clinical Trials. Current Topics in Medicinal Chemistry vol. 17 620-628.
19. Torres, M. D. T., Cao, J., Franco, O. L., Lu, T. K. & De La Fuente-Nunez, C. Synthetic Biology and Computer-Based Frameworks for Antimicrobial Peptide Discovery. ACS Nano (2021) doi: 10.1021/acsnano.0c09509.
20. Porto, W. F. et al. In silico optimization of a guava antimicrobial peptide enables combinatorial exploration for peptide design. Nat Commun 9, (2018).
21. Cardoso, M. H. et al. A Computationally Designed Peptide Derived from Escherichia coli as a Potential Drug Template for Antibacterial and Antibiofilm Therapies. ACS Infect Dis 4, 1727-1736 (2018).
22. Pane, K. et al. Identification of Novel Cryptic Multifunctional Antimicrobial Peptides from the Human Stomach Enabled by a Computational-Experimental Platform. ACS Synth Biol 7, 2105-2115 (2018).
23. Lee, E. Y., Lee, M. W., Fulan, B. M., Ferguson, A. L. & Wong, G. C. L. What can machine learning do for antimicrobial peptides, and what can antimicrobial peptides do for machine learning? Interface Focus 7, (2017).
24. Müller, A. T., Hiss, J. A. & Schneider, G. Recurrent Neural Network Model for Constructive Peptide Design. J Chem Inf Model 58, 472-479 (2018).
25. Bionda, N. et al. Identification of novel cyclic lipopeptides from a positional scanning combinatorial library with enhanced antibacterial and antibiofilm activities. Eur J Med Chem 108, 354-363 (2016).
26. Wang, J. et al. High specific selectivity and Membrane-Active Mechanism of the synthetic centrosymmetric α-helical peptides with Gly-Gly pairs. Sci Rep 5, 15963 (2015).
27. Torres, M. D. T. et al. Structure-function-guided exploration of the antimicrobial peptide polybia-CP identifies activity determinants and generates synthetic therapeutic candidates. Commun Biol 1, 221 (2018).
28. Jiang, Z., Mant, C. T., Vasil, M. & Hodges, R. S. Role of positively charged residues on the polar and non-polar faces of amphipathic α-helical antimicrobial peptides on specificity and selectivity for Gram-negative pathogens. Chem Biol Drug Des 91, 75-92 (2018).
29. Oliva, R. et al. Exploring the role of unnatural amino acids in antimicrobial peptides. Sci Rep 8, 8888 (2018).
30. Ramamoorthy, A. et al. Deletion of All Cysteines in Tachyplesin I Abolishes Hemolytic Activity and Retains Antimicrobial Activity and Lipopolysaccharide Selective Binding. Biochemistry 45, 6529-6540 (2006).
31. Huertas, N. de J., Monroy, Z. J. R., Medina, R. F. & Castañeda, J. E. G. Antimicrobial Activity of Truncated and Polyvalent Peptides Derived from the FKCRRQWQWRMKKGLA Sequence against Escherichia coli ATCC 25922 and Staphylococcus aureus ATCC 25923. Molecules 22, (2017).
32. Qu, P. et al. The Central Hinge Link Truncation of the Antimicrobial Peptide Fowlicidin-3 Enhances Its Cell Selectivity without Antibacterial Activity Loss. Antimicrob Agents Chemother 60, 2798-2806 (2016).
33. Jiang, X. et al. Design and activity study of a melittin-thanatin hybrid peptide. AMB Express 9, (2019)
34. Deshayes, S. et al. Designing Hybrid Antibiotic Peptide Conjugates to Cross Bacterial Membranes. Bioconjug Chem 28, 793-804 (2017).
35. Klubthawee, N., Adisakwattana, P., Hanpithakpong, W., Somsri, S. & Aunpad, R. A novel, rationally designed, hybrid antimicrobial peptide, inspired by cathelicidin and aurein, exhibits membrane-active mechanisms against Pseudomonas aeruginosa. Sci Rep 10, 9117 (2020).
36. Ajish, C. et al. A novel hybrid peptide composed of LfcinB6 and KR-12-a4 with enhanced antimicrobial, anti-inflammatory and anti-biofilm activities. Sci Rep 12, 4365 (2022).
37. Perumal Samy, R., Stiles, B. G., Franco, O. L., Sethi, G. & Lim, L. H. K. Animal venoms as antimicrobial agents. Biochem Pharmacol 134, 127-138 (2017).
38. Fratini, F., Cilia, G., Turchi, B. & Felicioli, A. Insects, arachnids and centipedes venom: A powerful weapon against bacteria. A literature review. Toxicon 130, 91-103 (2017).
39. Ramírez-Carreto, S. et al. Gene cloning and functional characterization of four novel antimicrobial-like peptides from scorpions of the family Vaejovidae. Peptides (N.Y.) 34, 290-295 (2012).
40. Konno, K. et al. Anoplin, a novel antimicrobial peptide from the venom of the solitary wasp Anoplius samariensis. Biochimica et Biophysica Acta—Protein Structure and Molecular Enzymology 1550, 70-80 (2001).
41. Mendes, M. A., De Souza, B. M., Marques, M. R. & Palma, M. S. Structural and biological characterization of two novel peptides from the venom of the neotropical social wasp Agelaia pallipes pallipes. Toxicon 44, 67-74 (2004).
42. Konno, K. et al. Decoralin, a novel linear cationic α-helical peptide from the venom of the solitary eumenine wasp Oreumenes decoratus. Peptides (N.Y.) 28, 2320-2327 (2007).
43. Patocka, J., Nepovimova, E., Klimova, B. & Kuca*, Q. W. and K. Antimicrobial Peptides: Amphibian Host Defense Peptides. Current Medicinal Chemistry vol. 25 1-21 Preprint at https://doi.org/http://dx.doi.org/10.2174/0929867325666180713125314 (2018).
44. Simmaco, M. et al. Temporins, Antimicrobial Peptides from the European Red Frog Rana temporaria. Eur J Biochem 242, 788-792 (1996).
45. Krugliak, M. et al. Antimalarial Activities of Dermaseptin S4 Derivatives. Antimicrob Agents Chemother 44, 2442 LP-2451 (2000).
46. Huang, L. et al. Dermaseptin-PH: A novel peptide with antimicrobial and anticancer activities from the skin secretion of the south American orange-legged leaf frog, pithecopus (phyllomedusa) hypochondrialis. Molecules 22, (2017).
47. Harjunpää, I. et al. Comparison of synthesis and antibacterial activity of temporin A. FEBS Lett 449, 187-190 (1999).
48. Simmaco, M. et al. Temporins, Antimicrobial Peptides from the European Red Frog Rana temporaria. Eur J Biochem 242, 788-792 (1996).
49. Chepurny, O. G. et al. Chimeric peptide EP45 as a dual agonist at GLP-1 and NPY2R receptors. Sci Rep 8, 1-14 (2018).
50. Tan, T. et al. High specific selectivity and membrane-active mechanism of synthetic cationic hybrid antimicrobial peptides based on the peptide FV7. Int J Mol Sci 18, (2017).
51. Jindal, H. M. et al. Antimicrobial activity of novel synthetic peptides derived from indolicidin and ranalexin against Streptococcus pneumoniae. PLOS One 10, 1-23 (2015).
52. Zelezetsky, I. & Tossi, A. Alpha-helical antimicrobial peptides-Using a sequence template to guide structure-activity relationship studies. Biochim Biophys Acta Biomembr 1758, 1436-1449 (2006).
53. Pedron, C. N. et al. Novel designed VmCT1 analogs with increased antimicrobial activity. Eur J Med Chem 126, (2017).
54. Mendes, M. A., De Souza, B. M., Marques, M. R. & Palma, M. S. Structural and biological characterization of two novel peptides from the venom of the neotropical social wasp Agelaia pallipes pallipes. Toxicon 44, 67-74 (2004).
55. Reddy Chichili, V. P., Kumar, V. & Sivaraman, J. Linkers in the structural biology of protein-protein interactions. Protein Science 22, 153-167 (2013).
56. Silva, A. F. et al. Effects of the angiotensin II Ala-scan analogs in erythrocytic cycle of Plasmodium falciparum (in vitro) and Plasmodium gallinaceum (ex vivo). Exp Parasitol 153, (2015).
57. Torres, M. D. T. et al. Peptide Design Enables Reengineering of an Inactive Wasp Venom Peptide into Synthetic Antiplasmodial Agents. ChemistrySelect 3, 5859-5863 (2018).
58. Torres, M. D. T. et al. The wasp venom antimicrobial peptide polybia-CP and its synthetic derivatives display antiplasmodial and anticancer properties. Bioeng Transl Med (2020) doi: 10.1002/btm2.10167.
59. Gaspar, D., Salomé Veiga, A. & Castanho, M. A. R. B. From antimicrobial to anticancer peptides. A review. Front Microbiol 4, 1-16 (2013).
60. Liu, X. Mechanism of Anticancer Effects of Antimicrobial Peptides. Journal of Fiber Bioengineering and Informatics 8, 25-36 (2015).
61. Huang, Y., Feng, Q., Yan, Q., Hao, X. & Chen, Y. Alpha-Helical Cationic Anticancer Peptides: A Promising Candidate for Novel Anticancer Drugs. Mini-Reviews in Medicinal Chemistry 15, 73-81 (2015).
62. Pedron, C. N. et al. Novel designed VmCT1 analogs with increased antimicrobial activity. Eur J Med Chem 126, 456-463 (2017).
63. Munk, J. K. et al. Synthetic analogs of anoplin show improved antimicrobial activities. Journal of Peptide Science 19, 669-675 (2013).
64. Torres, M. D. T. et al. Antimicrobial activity of leucine-substituted decoralin analogs with lower hemolytic activity. Journal of Peptide Science 23, 818-823 (2017).
65. Harjunpää, I. et al. Comparison of synthesis and antibacterial activity of temporin A. FEBS Lett 449, 187-190 (1999).
66. Heinig, M. & Frishman, D. STRIDE: a web server for secondary structure assignment from known atomic coordinates of proteins. Nucleic Acids Res 32, W500-W502 (2004).
67. Humphrey, W., Dalke, A. & Schulten, K. VMD: Visual molecular dynamics. J Mol Graph 14, 33-38 (1996).
68. Jafari, M. et al. Serum Stability and Physicochemical Characterization of a Novel Amphipathic Peptide C6M1 for SIRNA Delivery. PLOS One 9, e97797 (2014).
69. Maxian, T., Gerlitz, L., Riedl, S., Rinner, B. & Zweytick, D. Effect of L- to D-Amino Acid Substitution on Stability and Activity of Antitumor Peptide RDP215 against Human Melanoma and Glioblastoma. Int J Mol Sci 22, 8469 (2021).
70. Qiu, S. et al. Antimicrobial activity and stability of protonectin with D-amino acid substitutions. Journal of Peptide Science 23, 392-402 (2017).
71. Torres, M. D. T. et al. Decoralin Analogs with Increased Resistance to Degradation and Lower Hemolytic Activity. ChemistrySelect 2, 18-23 (2017).
72. Miller, C. C. & Walker, J. The Stokes-Einstein law for diffusion in solution. Proceedings of the Royal Society of London. Series A, Containing Papers of a Mathematical and Physical Character 106, 724-749 (1924).
73. Torres, M. D. T. et al. Coatable and Resistance-Proof Ionic Liquid for Pathogen Eradication. ACS Nano 15, 966-978 (2021).
74. Silva, O. N. et al. Repurposing a peptide toxin from wasp venom into antiinfectives with dual antimicrobial and immunomodulatory properties. Proc Natl Acad Sci USA 117, 26936-26945 (2020).
75. Cândido, E. S. et al. Short Cationic Peptide Derived from Archaea with Dual Antibacterial Properties and Anti-Infective Potential. ACS Infect Dis 5, 1081-1086 (2019).
76. Kyriakidis, I., Vasileiou, E., Pana, Z. D. & Tragiannidis, A. Acinetobacter baumannii Antibiotic Resistance Mechanisms. Pathogens 10, 373 (2021).
77. Torres, M. D. T. et al. Decoralin Analogs with Increased Resistance to Degradation and Lower Hemolytic Activity. ChemistrySelect 2, 18-23 (2017).
78. Mercer, D. K. et al. Antimicrobial Susceptibility Testing of Antimicrobial Peptides to Better Predict Efficacy. Front Cell Infect Microbiol 10, 326 (2020).
79. Koo, H. B. & Seo, J. Antimicrobial peptides under clinical investigation. Peptide Science 111, (2019).
80. Sikora, K. et al. Counter-ion effect on antistaphylococcal activity and cytotoxicity of selected antimicrobial peptides. Amino Acids 50, 609-619 (2018).
81. Cesaro, A., Torres, M. & de la Fuente-Nunez, C. Methods for the design and characterization of peptide antibiotics. in Methods in Enzymology (Academic Press, 2022). doi: 10.1016/bs.mie.2021.11.003.
82. Torres, M. D. T., Melo, M. C. R., Crescenzi, O., Notomista, E. & de la Fuente-Nunez, C. Mining for encrypted peptide antibiotics in the human proteome. Nat Biomed Eng 6, 67-75 (2022).
83. Silva, A. F. et al. Angiotensin II-derived constrained peptides with antiplasmodial activity and suppressed vasoconstriction. Sci Rep 7, 1-10 (2017).
84. Fani, S. et al. Anticancer activity of a monobenzyltin complex C1 against MDA-MB-231 cells through induction of Apoptosis and inhibition of breast cancer stem cells. Sci Rep 6, 38992 (2016).
85. Krishan, A. Rapid flow cytofluorometric analysis of mammalian cell cycle by propidium iodide staining. J Cell Biol 66, 188 LP-193 (1975).
86. Pedron, C. N. et al. Anticancer activity of VmCT1 analogs against MCF-7 cells. Chem Biol Drug Des 91, 588-596 (2018).
87. Torres, M. D. T. et al. Natural and redesigned wasp venom peptides with selective antitumoral activity. Beilstein Journal of Organic Chemistry 14, 1693-1703 (2018).
88. Shalel, S., Streichman, S. & Marmur, A. The mechanism of hemolysis by surfactants: Effect of solution composition. J Colloid Interface Sci 252, 66-76 (2002).
89. Love, L. H. The hemolysis of human erythrocytes by sodium dodecyl sulfate. J Cell Comp Physiol 36, 133-148 (2005).
90. Best, R. B. et al. Optimization of the Additive CHARMM All-Atom Protein Force Field Targeting Improved Sampling of the Backbone φ, ψ and Side-Chain χ1 and χ2 Dihedral Angles. J Chem Theory Comput 8, 3257-3273 (2012).
91. Jorgensen, W. L., Chandrasekhar, J., Madura, J. D., Impey, R. W. & Klein, M. L. Comparison of simple potential functions for simulating liquid water. J Chem Phys 79, 926-935 (1983).
92. Essmann, U. et al. A smooth particle mesh Ewald method. J Chem Phys 103, 8577-8593 (1995).
93. Jo, S., Kim, T., Iyer, V. G. & Im, W. CHARMM-GUI: A web-based graphical user interface for CHARMM. J Comput Chem 29, 1859-1865 (2008).
94. Lee, K. et al. Antibiotic activity and structural analysis of the scorpion-derived antimicrobial peptide IsCT and its analogs. Biochem Biophys Res Commun 323, 712-719 (2004).
95. Batista Martins, D. et al. Protonectin peptides target lipids, act at the interface and selectively kill metastatic breast cancer cells while preserving morphological integrity. J Colloid Interface Sci 601, 517-530 (2021).
96. Guerra, M. E. R. et al. MD simulations and multivariate studies for modeling the antileishmanial activity of peptides. Chem Biol Drug Des 90, 501-510 (2017).
97. Uggerhøj, L. E. et al. Rational Design of Alpha-Helical Antimicrobial Peptides: Do's and Don'ts. ChemBioChem 16, 242-253 (2015).
98. Manzo, G. et al. Minor sequence modifications in temporin B cause drastic changes in antibacterial potency and selectivity by fundamentally altering membrane activity. Sci Rep 9, 1385 (2019).
99. Krivov, G. G., Shapovalov, M. v. & Dunbrack, R. L. Improved prediction of protein side-chain conformations with SCWRL4. Proteins: Structure, Function, and Bioinformatics 77, 778-795 (2009).
100. Berendsen, H. J. C., Postma, J. P. M., van Gunsteren, W. F., DiNola, A. & Haak, J. R. Molecular dynamics with coupling to an external bath. J Chem Phys 81, 3684-3690 (1984).
101. Parrinello, M. & Rahman, A. Polymorphic transitions in single crystals: A new molecular dynamics method. J Appl Phys 52, 7182-7190 (1981).
102. Hoover, W. G. Canonical dynamics: Equilibrium phase-space distributions. Phys Rev A (Coll Park) 31, 1695-1697 (1985).
103. Hess, B., Bekker, H., Berendsen, H. J. C. & Fraaije, J. G. E. M. LINCS: A linear constraint solver for molecular simulations. J Comput Chem 18, 1463-1472 (1997).
104. Torres, M. D. T. et al. Coatable and Resistance-Proof Ionic Liquid for Pathogen Eradication. ACS Nano 15, 966-978 (2021).
105. Torres, M. D., Melo, M. C., Crescenzi, O., Notomista, E. & de la Fuente-Nunez, C. Mining for encrypted peptide antibiotics in the human proteome. Nat Biomed Eng (2021) doi: 10.1038/s41551-021-00801-1.
106. Torres, M. D. T. et al. Coatable and Resistance-Proof Ionic Liquid for Pathogen Eradication. ACS Nano 15, 966-978 (2021).
107. Gautier, R., Douguet, D., Antonny, B. & Drin, G. HELIQUEST: A web server to screen sequences with specific α-helical properties. Bioinformatics 24, 2101-2102 (2008).

1. A method for preparing a hybrid antimicrobial peptide comprising:
selecting a parent peptide having antimicrobial activity and a portion that represents a helical structure when contacted with a cell membrane bilayer;
selecting a template peptide having antimicrobial activity and a portion that represents a helical structure when contacted with a cell membrane bilayer;
combining a hydrophilic face of the helical structure of the parent peptide with the hydrophobic face of the helical structure of the templated peptide, or combining a hydrophobic face of the helical structure of the parent peptide with the hydrophilic face of the helical structure of the template peptide, thereby forming a hybrid helical structure, wherein the hybrid antimicrobial peptide comprises the hybrid helical structure.

2. The method according to claim 1, wherein the selection of the template peptide includes assessing the amphiphilicity, antimicrobial activity, or hemolytic activity of the template peptide.

3. The method according to claim 1, further comprising aligning the size of the hydrophobic face of the hybrid helical structure with the size of the hydrophilic face of the hybrid helical structure.

4. The method according to claim 3, wherein the aligning comprises adding one or more amino acid residues to an interface between the hydrophobic face of the hybrid helical structure and the hydrophilic face of the hybrid helical structure in order to provide a standardized amino acid length.

5. The method of claim 4, wherein the aligning comprises adding one or more glycine residues to an interface between the hydrophobic face of the hybrid helical structure and the hydrophilic face of the hybrid helical structure in order to provide the standardized amino acid length.

6. The method according to claim 3, wherein the standardized amino acid length corresponds to the number of amino acids of the parent peptide.

7. The method according to claim 1, wherein the parent peptide is VmCT1.

8. The method according to claim 1, wherein the template peptide is anoplin, protonectin, decoralin, or temporin A.

9. A hybrid antimicrobial peptide that is produced by the method according to claim 1.

10. A hybrid antimicrobial peptide comprising a hybrid helical portion comprising a combination of a hydrophobic face of a parent antimicrobial peptide and a hydrophilic face of a template antimicrobial peptide, or a combination of a combination of a hydrophilic face of the parent antimicrobial peptide and a hydrophobic face of the template antimicrobial peptide.

11. The hybrid antimicrobial peptide according to claim 10 wherein the hybrid helical portion comprises one or more introduced spacer amino acids for aligning by size the hydrophobic face with the hydrophilic face.

12. The hybrid antimicrobial peptide according to claim 10, wherein the parent antimicrobial peptide is VmCT1.

13. The hybrid antimicrobial peptide according to claim 10, wherein the template antimicrobial peptide is anoplin, protonectin, decoralin, or temporin A.

14. A method for treating a microbial infection in a subject comprising administering to the subject a hybrid antimicrobial peptide that is produced by the method according to claim 1.

15. A method for reducing a population of microbial organisms comprising contacting one or more of the microbial organisms with a hybrid antimicrobial peptide that is produced by the method according to claim 1.

16. A method for treating a microbial infection in a subject comprising administering to the subject a hybrid antimicrobial peptide according to claim 9.

17. A method for reducing a population of microbial organisms comprising contacting one or more of the microbial organisms with a hybrid antimicrobial peptide according to claim 9.
